(19) United States
(12) Patent Application Publication
(10) Pub. No.: US 2007/0128117 A1
(43) Pub. Date: Jun. 7, 2007

(54) Ultrasound contrast agents and process for the preparation thereof

(75) Inventors: Thierry Bettinger, Peillonnex (FR); Philippe Bussat, Feigeres (FR)

Correspondence Address: BRACCO RESEARCH USA INC., 305- COLLEGE ROAD EAST, PRINCETON, NJ, 08540, US

(73) Assignee: Bracco International B.V., Amsterdam (NL)

(21) Appl. No.: 11/641,289

(22) Filed: Dec. 19, 2006

Related U.S. Application Data
(63) Continuation-in-part of application No. 11/202,008, filed on Aug. 11, 2005.
Application No. 11/202,008 is a continuation-in-part of application No. 10/544,123, filed on Aug. 2, 2005.

(30) Foreign Application Priority Data
Feb. 3, 2004 (IB) PCT/IB04/00243
Feb. 4, 2003 (EP) EP03002375.8

(51) Int. Cl. A61K 49/22; A61K 39/00

(52) U.S. Cl. 424/9.52; 424/178.1

(57) ABSTRACT

Method for preparing a lyophilized matrix and, upon reconstitution of the same, a respective injectable contrast agent comprising a liquid aqueous suspension of gas-filled microbubbles stabilized predominantly by a phospholipid and comprising a ligand agent. The method comprises preparing an emulsion from an aqueous medium, comprising a phospholipid and a water immiscible organic solvent. A suspension of a compound comprising the ligand agent or a precursor thereof is then added to emulsion. The emulsion is then freeze-dried and subsequently reconstituted in an aqueous suspension of gas-filled microbubbles.

### CROSS REFERENCE TO RELATED APPLICATIONS

This application is a continuation-in-part of co-pending U.S. application, U.S. Ser. No. 11/202,008, filed Aug. 11, 2005, which is a continuation-in-part of co-pending U.S. application, U.S. Ser. No. 10/544,123, filed Aug. 2, 2005, which is the national stage application of international application PCT/IB2004/000243, filed Feb. 3, 2004, which claims priority to and the benefit of European application EP03002375.8, filed Feb. 4, 2003, all of which are hereby incorporated by reference.

### FIELD OF THE INVENTION

The present invention relates to a process for the preparation of a dry or lyophilized formulation useful for preparing a targeted gas-filled microbubbles usable in diagnostic imaging and to a process for preparing said gas filled microbubbles.

### BACKGROUND OF THE INVENTION

Rapid development of ultrasound contrast agents in the recent years has generated a number of different formulations, which are useful in ultrasound imaging of organs and tissue of human or animal body. These agents are designed to be used primarily as intravenous or intra-arterial injectables in conjunction with the use of medical echographic equipment which employs for example, B-mode image formation (based on the spatial distribution of backscatter tissue properties) or Doppler signal processing (based on Continuous Wave or pulsed Doppler processing of ultrasonic echoes to determine blood or liquid flow parameters).

A class of injectable formulations useful as ultrasound contrast agents includes suspensions of gas bubbles having a diameter of few microns dispersed in an aqueous medium.

Use of suspensions of gas bubbles in carrier liquid, as efficient ultrasound reflectors is well known in the art. The development of microbubble suspensions as echopharmaceuticals for enhancement of ultrasound imaging followed early observations that rapid intravenous injections of aqueous solutions can cause dissolved gases to come out of solution by forming bubbles. Due to their substantial difference in acoustic impedance relative to blood, these intravascular gas bubbles were found to be excellent reflectors of ultrasound. The injection of suspensions of gas bubbles in a carrier liquid into the blood stream of a living organism strongly reinforces ultrasonic echography imaging, thus enhancing the visualisation of internal organs. Since imaging of organs and deep seated tissues can be crucial in establishing medical diagnosis, a lot of effort has been devoted to the development of stable suspensions of highly concentrated gas bubbles which at the same time would be simple to prepare and administer, would contain a minimum of inactive species and would be capable of long storage and simple distribution.

The simple dispersion of free gas bubbles in the aqueous medium is however of limited practical interest, since these bubbles are in general not stable enough to be useful as ultrasound contrast agents.

Interest has accordingly been shown in methods of stabilising gas bubbles for echography and other ultrasonic studies, for example using emulsifiers, oils, thickeners or sugars, or by entrapping or encapsulating the gas or a precursor thereof in a variety of systems. These stabilized gas bubbles are generally referred to in the art as “microvesicles”, and may be divided into two main categories.

A first category of stabilized bubbles or microvesicles is generally referred to in the art as “microbubbles” and includes aqueous suspensions in which the bubbles of gas are bounded at the gas/liquid interface by a very thin envelope involving a surfactant (i.e. an amphiphilic material) disposed at the gas to liquid interface. A second category of microvesicles is generally referred to in the art as “microballoons” or “microcapsules” and includes suspensions in which the bubbles of gas are surrounded by a solid material envelope formed of natural or synthetic polymers. Examples of microballoons and of the preparation thereof are disclosed, for instance, in European patent application EP 0458745. Another kind of ultrasound contrast agent includes suspensions of porous microparticles of polymers or other solids, which carry gas bubbles entrapped within the pores of the microparticles. The present invention is particularly concerned with contrast agents for diagnostic imaging including an aqueous suspension of gas microbubbles, i.e. microvesicles which are stabilized essentially by a layer of amphiphilic material.

Microbubbles suspensions are typically prepared by contacting powdered amphiphilic materials, e.g. freeze-dried preformed liposomes or freeze-dried or spray-dried phospholipid suspensions, with air or other gas and then with aqueous carrier, agitating to generate a microbubble suspension which must then be administered shortly after its preparation.

Examples of aqueous suspensions of gas microbubbles and preparation thereof can be found for instance in U.S. Pat. Nos. 5,271,928, 5,445,813, 5,413,774, 5,556,610, 5,597,549, 5,827,504.

WO94/011940 and WO97/29783 disclose alternative processes for preparing gas microbubbles suspensions.

Furthermore, interest has also been shown towards compositions comprising targeted gas-filled microvesicles, as disclosed for instance in U.S. Pat. Nos. 5,531,980, 6,131,819 and WO 98/18501.

The preparation method developed by the Applicant allows effectively incorporating a ligand agent, such as a targeting ligand, in particular an antibody, fragment or precursor thereof, into a formulation of gas-filled microbubbles.

The process of the invention further allows to obtain targeted gas-filled microbubbles with a controlled size distribution and, if desired, relatively small diameter.

### SUMMARY OF THE INVENTION

An aspect of the invention relates to a method for preparing an aqueous suspension of gas-filled microbubbles comprising a ligand agent, which comprises the steps of:
a) preparing an aqueous-organic emulsion comprising i) an aqueous medium, ii) an organic solvent substantially immiscible with water, iii) an amphiphilic material comprising a phospholipid and iv) a lyoprotecting agent;
b) adding a precursor of said ligand agent to said aqueous-organic emulsion;
c) converting said precursor into said ligand agent;
d) lyophilizing said mixture, to obtain a lyophilized matrix;
e) contacting said lyophilized matrix with a biocompatible gas; and
f) reconstituting said lyophilized matrix by dissolving it in a physiologically acceptable aqueous carrier liquid, to obtain said suspension of gas-filled microbubbles.

A further aspect of the invention relates to a method for preparing a lyophilized precursor of gas-filled microbubbles comprising a ligand agent comprising the steps of:
a) preparing an aqueous-organic emulsion comprising i) an aqueous medium, ii) an organic solvent substantially immiscible with water, iii) an amphiphilic material comprising a phospholipid and iv) a lyoprotecting agent;
b) adding a precursor of said ligand agent to said aqueous-organic emulsion;
c) converting said precursor into said ligand agent;
d) lyophilizing said emulsified mixture, to obtain a lyophilized matrix.

Preferably said precursor of a ligand agent is an amphiphilic compound, preferably comprising a phospholipid, more preferably in combination with an hydrophilic spacer. Preferably, said ligand agent comprises a moiety of an affinity binding pair, such as avidin or streptavidin, or a targeting ligand, preferably an antibody or a fragment thereof.

Preferably, said precursor of the ligand agent is added to said aqueous-organic emulsion in the form of an aqueous micellar suspension of said ligand agent.

According to a preferred embodiment of the invention, when the ligand agent comprises a moiety of an affinity binding pair, the obtained microvesicles are subsequently associated with a targeting ligand comprising a complementary moiety of said affinity binding pair.

### DETAILED DESCRIPTION OF THE INVENTION

The term “ligand agent” includes any compound, moiety or residue having a ligand activity towards a complementary moiety, a tissue or a receptor. These include moieties capable of forming an affinity binding pair as well as targeting ligands.

The expression “moiety forming an affinity binding pair” includes any functional moiety having a high affinity and selectivity (binding) for a complementary moiety, said binding being typically non-covalent. Examples of affinity binding pairs are, for instance, avidin (or streptavidin) binding to complementary moiety biotin; protein A or G binding to complementary Fc-region of immunoglobulin; oligonucleotides binding with complementary sequences, e.g. Polydesoxyadenylic acid binding with Polydesoxythimidylic acid, or Polydesoxyguanylic acid binding with Polydesoxycytidylic acide; Ni-NTA (nitrilotriacetic acid, nickel salt) binding with complementary Poly histidine-tagged ligand; PDBA (phenyldiboronic acid) binding with SHA (salicylhydroxamic acid). Preferred affinity binding pairs are avidin or streptavidin and biotin. Any of the above binding pairs can be used, for instance, to bind a targeting ligand (comprising a moiety of the pair) to an amphiphilic molecule (comprising the complementary moiety).

The term “targeting ligand” includes any compound, moiety or residue having, or being capable to promote, a targeting activity towards tissues and/or receptors in vivo. Targets to which a targeting ligand may be associated include tissues such as, for instance, myocardial tissue (including myocardial cells and cardiomyocytes), membranous tissues (including endothelium and epithelium), laminae, connective tissue (including interstitial tissue) or tumors; blood clots; and receptors such as, for instance, cell-surface receptors for peptide hormones, neurotransmitters, antigens, complement fragments, immunoglobulins and cytoplasmic receptors for steroid hormones.

The term “precursor” of a ligand agent (or of a targeting ligand) includes any moiety or compound (preferably an amphiphilic compound) which can be converted into a ligand agent (or targeting ligand), including any precursor functionalized with a suitable reactive moiety (e.g. maleimide), which can be reacted with a corresponding complementary moiety (e.g. thiol) linked to a ligand compound (or targeting ligand). Examples of preferred precursors are functionalized phospholipids (e.g. PE), preferably comprising a spacer.

As used herein, the term “spacer” includes any moiety which can bind to an amphiphilic moiety, on one side, and to a ligand agent on the other side. Examples of suitable spacers are disclosed, for instance, in WO98/18501 and WO 96/40285. Preferred spacers are hydrophilic polymers, such as, for instance, polyethylenglycol (PEG) or polypropylenglycol (PPG). Preferably, said spacer is covalently bound by means of a reactive moiety, including those illustrated hereinafter for binding a ligand agent to an amphiphilic molecule.

The term “gas-filled microbubbles” includes aqueous suspensions in which the bubbles of gas are bounded at the gas/liquid interface by a very thin envelope (film) involving a stabilizing amphiphilic material disposed at the gas to liquid interface (sometimes referred to in the art as “evanescent” envelope). Microbubbles suspensions can be prepared by contacting a suitable precursor thereof, such as powdered amphiphilic materials (e.g. freeze-dried preformed liposomes or freeze-dried or spray-dried phospholipid solutions) with air or other gas and then with an aqueous carrier, while agitating to generate a microbubble suspension which can then be administered, preferably shortly after its preparation. Examples of aqueous suspension of gas microbubbles, of precursors and of preparation thereof are disclosed, for instance, in U.S. Pat. Nos. 5,271,928, 5,445,813, 5,413,774, 5,556,610, 5,597,549, 5,827,504 and WO 04/069284, which are here incorporated by reference in their entirety.

The term “lyophilized precursor of gas-filled microbubbles” includes any lyophilized or freeze-dried composition comprising an amphiphilic material which, upon reconstitution with an aqueous carrier in the presence of a gas, is capable of forming a suspension of gas-filled microbubbles.

The term “targeted gas-filled microbubbles” includes gas-filled microbubbles comprising at least one targeting ligand in their formulation.

The term “precursor of a targeted gas-filled microvesicle” includes any gas-filled microvesicle which can be converted into a targeted gas-filled microvesicle. Such precursor may include, for instance, gas-filled microvesicles including a suitable reactive moiety (e.g. maleimide or streptavidin), which can be reacted with a corresponding complementary reactive (e.g. thiol or biotin, respectively) linked to a targeting ligand.

The aqueous medium employed in the process of the invention is preferably a physiologically acceptable carrier. The term “physiologically acceptable” includes to any compound, material or formulation which can be administered, in a selected amount, to a patient without negatively affecting or substantially modifying its organism's healthy or normal functioning (e.g. without determining any status of unacceptable toxicity, causing any extreme or uncontrollable allergenic response or determining any abnormal pathological condition or disease status).

Suitable aqueous liquid carriers are water, typically sterile, pyrogen free water (to prevent as much as possible contamination in the intermediate lyophilized product), aqueous solutions such as saline (which may advantageously be balanced so that the final product for injection is not hypotonic), or aqueous solutions of one or more tonicity adjusting substances such as salts or sugars, sugar alcohols, glycols or other non-ionic polyol materials (eg. glucose, sucrose, sorbitol, mannitol, glycerol, polyethylene glycols, propylene glycols and the like).

The Organic Solvent

As used herein the term “substantially immiscible with water” referred to the organic solvent means that, when said solvent is admixed with water, two separate phases are formed. Water immiscible solvent are generally also known in the art as apolar or non-polar solvents, as opposed to polar solvents (such as water). Water immiscible solvents are in general substantially insoluble in water. For the purposes of the present invention, organic solvents suitable for being emulsified with the aqueous solvent are typically those solvents having a solubility in water of less than about 10 g/l. Preferably, the solubility of said solvent in water is of about 1.0 g/l or lower, more preferably about 0.2 g/l or lower and much more preferably about 0.01 g/l or lower. Particularly preferred solvents are those having a solubility in water of 0.001 g/l or lower. Particularly insoluble organic solvents (e.g. perfluorocarbons) may have a solubility down to about 1.0·10⁻⁶ g/l (e.g perfluorooctane, 1.66·10⁻⁶ g/l).

The organic solvent is preferably lyophilisable, i.e. said solvent has a sufficiently high vapour pressure at the lyophilization temperatures, e.g. between −30° C. and 0° C., to allow for an effective and complete evaporation/sublimation within acceptable times, e.g. 24-48 hours. Preferably, the vapour pressure of the organic solvent is higher than about 0.2 kPa at 25° C.

The organic solvent can be selected from a broad range of solvents and any chemical entity that is water-immiscible and lyophilisable, as indicated above, and being preferably liquid at room temperature (25° C.). If a solvent having a boiling point lower than room temperature is used, the vessel containing the emulsifying mixture can advantageously be cooled below the boiling point of said solvent, e.g. down to 5° C. or 0° C. As said solvent will be completely removed during the lyophilization step, no particular constraints exist except that it should not contain contaminants that cannot be removed through lyophilisation or that are not acceptable for use in an injectable composition.

Suitable organic solvents include but are not limited to alkanes, such as branched or, preferably, linear (C₅-C₁₀) alkanes, e.g. pentane, hexane, heptane, octane, nonane, decane; alkenes, such as (C₅-C₁₀) alkenes, e.g. 1-pentene, 2-pentene, 1-octene; cyclo-alkanes, such as (C₅-C₈)-cycloalkanes optionally substituted with one or two methyl groups, e.g. cyclopentane, cyclohexane, cyclooctane, 1-methyl-cyclohexane; aromatic hydrocarbons, such as benzene and benzene derivatives substituted by one or two methyl or ethyl groups, e.g. benzene, toluene, ethylbenzene, 1,2-dimethylbenzene, 1,3-dimethylbenzene; alkyl ethers and ketones such as di-butyl ether and di-isopropylketone; halogenated hydrocarbons or ethers, such as chloroform, carbon tetrachloride, 2-chloro-1-(difluoromethoxy)-1,1,2-trifluoroethane (enflurane), 2-chloro-2-(difluoromethoxy)-1,1,1-trifluoroethane (isoflurane), tetrachloro-1,1-difluoroethane, and particularly perfluorinated hydrocarbons or ethers, such as perfluoropentane, perfluorohexane, perfluoroheptane, perfluoromethylcyclohexane, perfluorooctane, perfluorononane, perfluorobenzene and perfluorodecalin, methylperfluorobutylether, methylperfluoroisobutylether, ethylperfluorobutylether, ethylperfluoroisobutylether; and mixtures thereof.

The amount of solvent is generally comprised from about 1% to about 50% by volume with respect to the amount of water used for the emulsion. Preferably said amount is from about 1% to about 20%, more preferably from about 2% to about 15% and even more preferably from about 5% to about 10%. If desired, a mixture of two or more of the above listed organic solvents can be used, the overall amount of organic solvent in the emulsifying mixture being within the above range.

Lyoprotective Agent

The term lyoprotective agent or “lyoprotectant” refers to a compound which, when included in a formulation to be lyophilized, will protect the chemical compounds from the deleterious effects of freezing and vacuumizing, such as those usually accompanying lyophilization, e.g. damage, adsorption and loss from vacuum utilized in lyophilization. In addition, after the lyophilization step, said lyoprotective agent preferably results in a solid matrix (“bulk”) which supports the lyophilized phospholipid.

The present invention is not limited to the use of a specific lyoprotectant, and examples of suitable lyoprotectants include, but are not limited to, carbohydrates such as the saccharides, mono-, di- or poly-saccharides, e.g. glucose, galactose, fructose, sucrose, trehalose, maltose, lactose, amylose, amylopectin, cyclodextrins, dextran, inuline, soluble starch, hydroxyethyl starch (HES), sugar alcohols e.g. mannitol, sorbitol and polyglycols such as polyethyleneglycols. A substantial list of agents with lyoprotective effects is given in Acta Pharm. Technol. 34(3), pp. 129-139 (1988), the content of which is incorporated herein by reference. Said lyoprotective agents can be used singularly or as mixtures of one or more compounds.

Preferred lyoprotectants include mannitol and polysaccharides such as dextrans (in particular those with molecular weights above 1500 daltons), inulin, soluble starch, hydroxyethyl starch and polyethyleneglycols, preferably of MW from about 1000 to about 30000 daltons, more preferably from 2000 to 8000 daltons (e.g. PEG4000).

Mixtures of mannitol or polysaccharides such as dextrans, inulin, soluble starch, hydroxyethyl starch with saccharides such as glucose, maltose, lactose, sucrose, trehalose and erythritol also provide excellent results.

Likewise, the present invention is not limited to any particular amount of lyoprotectant used. However the optimal weight concentration of lyoprotective agents in the emulsion prior to the lyophilisation is comprised between about 1 and about 25%, preferably between about 2 and about 20%, and even more preferably between about 5 and about 10%.

A higher amount can be employed if it is also necessary to provide a desired “bulk” to the lyophilized product.

The lyoprotective agent is preferably added to the aqueous-organic mixture before emulsification of the same and in this case the emulsification of the aqueous-organic mixture is thus carried out in the presence of the lyoprotective agents. Alternatively, the lyoprotectant can be added to the aqueous-organic mixture after the emulsification thereof. In the first case, the lyoprotectant is preferably added to the aqueous medium, before admixing it with the organic solvent. If desired, it is also possible to combine the two, e.g. by adding part of the lyoprotective agent to the aqueous phase used for the preparation of the emulsion and part to the thus obtained emulsion. If desired, also cryoprotective agents, such as glycerol, can further be added to the emulsion for protecting the chemical compounds from the deleterious effects of freezing.

Phospholipids

According to the present description and claims, the term phospholipid is intended to encompass any amphiphilic phospholipidic compound the molecules of which are capable of forming a film of material (typically in the form of a mono-molecular layer) at the gas-water boundary interface in the final microbubbles suspension. Accordingly, these material are also referred to in the art as “film-forming phospholipids”. Similarly, in the emulsified mixture, these amphiphilic compounds are typically disposed at the interface between the aqueous medium and the organic solvent substantially insoluble in water, thus stabilizing the emulsified solvent microdroplets. The film formed by these compounds at the gas-water or water-solvent interface can be either continuous or discontinuous. In the latter case, the discontinuities in the film should not however be such as to impair the stability (e.g. pressure resistance, resistance to coalescence, etc.) of the suspended microbubbles or of the emulsified microdroplets, respectively.

The term “amphiphilic compound” as used herein includes compounds having a molecule with a hydrophilic polar head portion (e.g. a polar or ionic group), capable of interacting with an aqueous medium, and a hydrophobic organic tail portion (e.g. a hydrocarbon chain), capable of interacting with e.g. an organic solvent. These compounds thus generally act as “surface active agent”, i.e. compounds which are capable of stabilizing mixtures of otherwise generally immiscible materials, such as mixtures of two immiscible liquids (e.g. water and oil), mixtures of liquids with gases (e.g. gas microbubbles in water) or mixtures of liquids with insoluble particles (e.g. metal nanoparticles in water).

Amphiphilic phospholipid compounds typically contain at least one phosphate group and at least one, preferably two, lipophilic long-chain hydrocarbon group.

Examples of suitable phospholipids include esters of glycerol with one or preferably two (equal or different) residues of fatty acids and with phosphoric acid, wherein the phosphoric acid residue is in turn bound to a hydrophilic group, such as choline (phosphatidylcholines—PC), serine (phosphatidylserines—PS), glycerol (phosphatidylglycerols—PG), ethanolamine (phosphatidylethanolamines—PE), inositol (phosphatidylinositol), and the like groups. Esters of phospholipids with only one residue of fatty acid are generally referred to in the art as the “lyso” forms of the phospholipid. Fatty acids residues present in the phospholipids are in general long chain aliphatic acids, typically containing from 12 to 24 carbon atoms, preferably from 14 to 22; the aliphatic chain may contain one or more unsaturations or is preferably completely saturated. Examples of suitable fatty acids included in the phospholipids are, for instance, lauric acid, myristic acid, palmitic acid, stearic acid, arachidic acid, behenic acid, oleic acid, linoleic acid, and linolenic acid. Preferably, saturated fatty acids such as myristic acid, palmitic acid, stearic acid and arachidic acid are employed.

Further examples of phospholipid are phosphatidic acids, i.e. the diesters of glycerol-phosphoric acid with fatty acids; sphingolipids such as sphingomyelins, i.e. those phosphatidylcholine analogs where the residue of glycerol diester with fatty acids is replaced by a ceramide chain; cardiolipins, i.e. the esters of 1,3-diphosphatidylglycerol with a fatty acid; glycolipids such as gangliosides GM1 (or GM2) or cerebrosides; glucolipids; sulfatides and glycosphingolipids.

As used herein, the term phospholipids include either naturally occurring, semisynthetic or synthetically prepared products that can be employed either singularly or as mixtures.

Examples of naturally occurring phospholipids are natural lecithins (phosphatidylcholine (PC) derivatives) such as, typically, soya bean or egg yolk lecithins.

Examples of semisynthetic phospholipids are the partially or fully hydrogenated derivatives of the naturally occurring lecithins. Preferred phospholipids are fatty acids di-esters of phosphatidylcholine, ethylphosphatidylcholine, phosphatidylglycerol, phosphatidic acid, phosphatidylethanolamine, phosphatidylserine or of sphingomyelin.

Examples of preferred phospholipids are, for instance, dilauroyl-phosphatidylcholine (DLPC), dimyristoyl-phosphatidylcholine (DMPC), dipalmitoyl-phosphatidylcholine (DPPC), diarachidoyl-phosphatidylcholine (DAPC), distearoyl-phosphatidylcholine (DSPC), dioleoyl-phosphatidylcholine (DOPC), 1,2 Distearoyl-sn-glycero-3-Ethylphosphocholine (Ethyl-DSPC), dipentadecanoyl-phosphatidylcholine (DPDPC), 1-myristoyl-2-palmitoyl-phosphatidylcholine (MPPC), 1-palmitoyl-2-myristoyl-phosphatidylcholine (PMPC), 1-palmitoyl-2-stearoyl-phosphatidylcholine (PSPC), 1-stearoyl-2-palmitoyl-phosphatidylcholine (SPPC), ), 1-palmitoyl-2-oleylphosphatidylcholine (POPC), 1-oleyl-2-palmitoyl-phosphatidylcholine (OPPC), dilauroyl-phosphatidylglycerol (DLPG) and its alkali metal salts, diarachidoylphosphatidyl-glycerol (DAPG) and its alkali metal salts, dimyristoylphosphatidylglycerol (DMPG) and its alkali metal salts, dipalmitoylphosphatidylglycerol (DPPG) and its alkali metal salts, distearoylphosphatidylglycerol (DSPG) and its alkali metal salts, dioleoyl-phosphatidylglycerol (DOPG) and its alkali metal salts, dimyristoyl phosphatidic acid (DMPA) and its alkali metal salts, dipalmitoyl phosphatidic acid (DPPA) and its alkali metal salts, distearoyl phosphatidic acid (DSPA), diarachidoylphosphatidic acid (DAPA) and its alkali metal salts, dimyristoyl-phosphatidylethanolamine (DMPE), dipalmitoylphosphatidylethanolamine (DPPE), distearoyl phosphatidyl-ethanolamine (DSPE), dioleylphosphatidyl-ethanolamine (DOPE), diarachidoyl-phosphatidylethanolamine (DAPE), dilinoleylphosphatidylethanolamine (DLPE), dimyristoyl phosphatidylserine (DMPS), diarachidoyl phosphatidylserine (DAPS), dipalmitoyl phosphatidylserine (DPPS), distearoylphosphatidylserine (DSPS), dioleoylphosphatidylserine (DOPS), dipalmitoyl sphingomyelin (DPSP), and distearoylsphingomyelin (DSSP).

The term phospholipid further includes modified phospholipid, e.g. phospholipids where the hydrophilic group is in turn bound to another hydrophilic group. Examples of modified phospholipids are phosphatidylethanolamines modified with polyethylenglycol (PEG), i.e. phosphatidylethanolamines where the hydrophilic ethanolamine moiety is linked to a PEG molecule of variable molecular weight e.g. from 300 to 5000 daltons, such as DPPE-PEG or DSPE-PEG, i.e. DPPE (or DSPE) having a PEG polymer attached thereto. For example, DPPE-PEG2000 refers to DPPE having attached thereto a PEG polymer having a mean average molecular weight of about 2000. As explained in detail in the following, these PEG-modified phospholipids are preferably used in combination with non-modified phospholipids.

Both neutral and charged phospholipids can satisfactorily be employed in the process of the present invention, as well as mixtures thereof. As used herein and in the prior art, the term “charged” in relation with “phospholipids” means that the individual phospholipid molecules have an overall net charge, be it positive or, more frequently, negative.

Examples of phospholipids bearing an overall negative charge are derivatives, in particular fatty acid di-esters, of phosphatidylserine, such as DMPS, DPPS, DSPS; of phosphatidic acid, such as DMPA, DPPA, DSPA; of phosphatidylglycerol such as DMPG, DPPG and DSPG. Also modified phospholipids, in particular PEG-modified phosphatidylethanolamines, such as DMPE-PEG750, DMPE-PEG1000, DMPE-PEG2000, DMPE-PEG3000, DMPE-PEG4000, DMPE-PEG5000, DPPE-PEG750, DPPE-PEG1000, DPPE-PEG2000, DPPE-PEG3000, DPPE-PEG4000, DPPE-PEG5000, DSPE-PEG750, DSPE-PEG1000, DSPE-PEG2000, DSPE-PEG3000, DSPE-PEG4000, DSPE-PEG5000, DAPE-PEG750, DAPE-PEG1000, DAPE-PEG2000, DAPE-PEG3000, DAPE-PEG4000 or DAPE-PEG5000 can be used as negatively charged molecules. Also the lyso- form of the above cited phospholipids, such as lysophosphatidylserine derivatives (e.g. lyso-DMPS, -DPPS or -DSPS), lysophosphatidic acid derivatives (e.g. lyso-DMPA, -DPPA or -DSPA) and lysophosphatidylglycerol derivatives (e.g. lyso-DMPG, -DPPG or -DSPG), can advantageously be used as negatively charged compound.

Examples of phospholipids bearing an overall positive charge are derivatives of ethylphosphatidylcholine, in particular esters of ethylphosphatidylcholine with fatty acids, such as 1,2-Distearoyl-sn-glycero-3-Ethylphosphocholine (Ethyl-DSPC or DSEPC), 1,2-Dipalmitoyl-sn-glycero-3-Ethylphosphocholine (Ethyl-DPPC or DPEPC).

According to preferred embodiments, blends of two or more phospholipids, at least one with a neutral charge and at least one with an overall net charge, are employed. More preferably, blends of two or more phospholipids, at least one with neutral and at least one with negative charge are employed. The amount of charged phospholipid, may vary from about 95% to about 5% by weight, with respect to the total amount of phospholipid, preferably from 80% to 20% by weight. The presence of at least minor amounts, such as 5% to 20% by wt. with respect to the total weight of phospholipid, of a (negatively) charged phospholipid may help preventing aggregation of bubbles or emulsion droplets. It is however possible to use a single phospholipid, neutral or charged, or a blend of two or more phospholipids, all neutral or all with an overall net charge.

The amount of phospholipid is generally comprised between about 0.005 and about 1.0% by weight with respect to the total weight of the emulsified mixture. Larger amounts might of course be employed but considering that the end product is an injectable contrast agent, it is preferred not to use excess of additives unless strictly necessary to provide for a stable and suitable product. In general, by using an amount of phospholipid larger than that indicated as the upper limit of the above range, essentially no or a very negligible improvement is observed in terms of bubble population, bubble size distribution, and bubble stability. Typically, higher amounts of phospholipid are required when higher volumes of organic solvent are used. Thus, when the volume of organic solvent amounts to about 50% the volume of the water phase, an amount of about 1% w/w of phospholipid can advantageously be added to the emulsion. Preferably the amount of phospholipid is comprised between 0.01 and 1.0% by weight with respect to the total weight of the emulsified mixture and more preferably between about 0.05% and 0.5% by weight.

As mentioned before, the microbubbles produced according to the process of the invention are stabilized predominantly by a phospholipid, as above defined. In particular, the envelope surrounding the gas filled microbubbles preferably comprises more than 50% (w/w) of a phospholipid material as above defined, more preferably at least 80%, and much more preferably at least 90%. According to certain embodiments, the substantial totality of the stabilizing envelope of the microbubbles is formed by a phospholipid.

Other amphiphilic materials can however be admixed with the phospholipids forming the stabilizing envelope of the gas-filled microbubbles, preferably in amounts of less than 50% of the total weight of the amphiphilic material contained in the initial emulsion.

Examples of suitable additional envelope-stabilizing amphiphilic materials include, for instance, lysolipids; fatty acids, such as palmitic acid, stearic acid, lauric acid, myristic acid, arachidic acid, arachidonic acid, behenic acid, oleic acid, linoleic acid or linolenic acid, and their respective salts with alkali or alkali metals; lipids bearing polymers, such as chitin, hyaluronic acid, polyvinylpyrrolidone or polyethylene glycol (PEG), also referred as “pegylated lipids”; lipids bearing sulfonated mono- di-, oligo- or polysaccharides; lipids with ether or ester-linked fatty acids; polymerized lipids; diacetyl phosphate; dicetyl phosphate; stearylamine; ceramides; polyoxyethylene fatty acid esters (such as polyoxyethylene fatty acid stearates); polyoxyethylene fatty alcohols; polyoxyethylene fatty alcohol ethers; polyoxyethylated sorbitan fatty acid esters; glycerol polyethylene glycol ricinoleate; ethoxylated soybean sterols; ethoxylated castor oil; ethylene oxide (EO) and propylene oxide (PO) block copolymers; sterol esters of sugar acids including cholesterol glucuronides, lanosterol glucoronides, 7-dehydrocholesterol glucoronide, ergosterol glucoronide, cholesterol gluconate, lanosterol gluconate, or ergosterol gluconate; esters of sugar acids and alcohols including lauryl glucoronide, stearoyl glucoronide, myristoyl glucoronide, lauryl gluconate, myristoyl gluconate, or stearoyl gluconate; esters of sugars with aliphatic acids including sucrose laurate, fructose laurate, sucrose palmitate, sucrose stearate, glucuronic acid, gluconic acid or polyuronic acid; esters of glycerol with (C₁₂-C₂₄), preferably (C₁₄-C₂₂) dicarboxylic fatty acids and their respective salts with alkali or alkali-metal salts, such as 1,2-dipalmitoyl-sn-3-succinylglycerol or 1,3-dipalmitoyl-2-succinylglycerol; saponins including sarsasapogenin, smilagenin, hederagenin, oleanolic acid, or digitoxigenin; long chain (C₁₂-C₂₄) alcohols, including n-decyl alcohol, lauryl alcohol, myristyl alcohol, cetyl alcohol, or n-octadecyl alcohol; 6-(5-cholesten-3β-yloxy)-1-thio-β-D-galactopyranoside; digalactosyldiglyceride; 6-(5-cholesten-3β-yloxy)hexyl-6-amino-6-deoxy-1-thio-β-D-galactopyranoside; 6-(5-cholesten-3β-yloxy)hexyl-6-amino-6-deoxyl-1-thio-β-D-mannopyranoside; 12-(((7′-diethylaminocoumarin-3-yl)carbonyl)methylamino)octadecanoic acid; N-[12-(((7′-diethylaminocoumarin-3-yl)carbonyl)-methylamino)octadecanoyl]-2-aminopalmitic acid; N-succinyldioleylphosphatidylethanolamine; 1-hexadecyl-2-palmitoylglycerophosphoethanolamine; palmitoylhomocysteine; alkylammonium salts comprising at least one (C₁₀-C₂₀), preferably (C₁₄-C₁₈), alkyl chain, such as, for instance, stearylammonium chloride, hexadecylammonium chloride, dimethyldioctadecylammonium bromide (DDAB), hexadecyltrimethylammonium bromide (CTAB); tertiary or quaternary ammonium salts comprising one or preferably two (C₁₀-C₂₀), preferably (C₁₄-C₁₈), acyl ester residue, such as, for instance, 1,2-distearoyl-3-trimethylammonium-propane (DSTAP), 1,2-dipalmitoyl-3-trimethylammonium-propane (DPTAP), 1,2-oleoyl-3-trimethylammonium-propane (DOTAP), 1,2-distearoyl-3-dimethylammonium-propane (DSDAP): and mixtures or combinations thereof.

Small amounts of fatty acids and lyso forms of the phospholipids may also form as degradation products of the original phospholipid products, e.g. as a consequence of heating the emulsion.

Preferred additional envelope-stabilizing amphiphilic materials are those compounds comprising one or two fatty acid residues in their molecule, in particular one or two linear (C₁₀-C₂₀)-acyl, preferably (C₁₄-C₁₈)-acyl chains, such as, for instance, the above listed fatty acids, their respective salts and derivatives.

Particularly preferred additional envelope-stabilizing amphiphilic materials are those compounds capable of conferring an overall net charge to the stabilizing envelope, i.e. compounds bearing an overall positive or negative net charge. Examples of suitable negatively of positively charged compounds are, for instance, lyso-phospholipids, i.e. the lyso- form of the above cited phospholipids, such as lysophosphatidylserine derivatives (e.g. lyso-DMPS, -DPPS or -DSPS), lysophosphatidic acid derivatives (e.g. lyso-DMPA, -DPPA or -DSPA) and lysophosphatidylglycerol derivatives (e.g. lyso-DMPG, -DPPG or -DSPG); bile acid salts such as cholic acid salts, deoxycholic acid salts or glycocholic acid salts; (C₁₂-C₂₄), preferably (C₁₄-C₂₂) fatty acid salts such as, for instance, palmitic acid salt, stearic acid salt, 1,2-dipalmitoyl-sn-3-succinylglycerol salt or 1,3-dipalmitoyl-2-succinylglycerol salt; alkylammonium salts with a halogen counter ion (e.g. chlorine or bromine) comprising at least one (C₁₀-C₂₀) alkyl chain, preferably (C₁₄-C₁₈) alkyl chain, such as, for instance stearylammonium chloride, hexadecylammonium chloride, dimethyldioctadecylammonium bromide (DDAB), hexadecyltrimethylammonium bromide (CTAB); tertiary or quaternary ammonium salts with a halogen counter ion (e.g. chlorine or bromine) comprising one or preferably two (C₁₀-C₂₀) acyl chain, preferably (C₁₄-C₁₈) acyl ester residue, such as, for instance, 1,2-distearoyl-3-trimethylammonium-propane (DSTAP), 1,2-dipalmitoyl-3-trimethylammonium-propane (DPTAP), 1,2-oleoyl-3-trimethylammonium-propane (DOTAP), 1,2-distearoyl-3-dimethylammonium-propane (DSDAP).

Particularly preferred combinations of amphiphilic materials comprise a phosphatydilcholine, preferably DSPC, in admixture with at least one component selected from the group consisting of DSPG, DPPG, DSPA, DPPA, stearic acid and palmitic acid.

Materials or substances which may serve as targeting ligands include, for example, but are not limited to proteins, including antibodies, antibody fragments, receptor molecules, receptor binding molecules, glycoproteins and lectins; peptides, including oligopeptides and polypeptides; peptidomimetics; saccharides, including mono and polysaccharides; vitamins; steroids, steroid analogs, hormones, cofactors, bioactive agents and genetic material, including nucleosides, nucleotides and polynucleotides. Preferably said targeting ligand is an antibody or a fragment thereof.

Examples of suitable ligand agents, targets and targeting ligands are disclosed, for instance, in international patent application WO 98/18501, which is herein incorporated by reference.

In a preferred embodiment, the ligand agent is bound to an amphiphilic molecule (which will then be associated to the stabilizing envelope of the microbubble, e.g. a phospholipid)through a covalent bond.

In such a case, the specific reactive moiety that needs to be present in the amphiphilic molecule, will depend on the corresponding reactive moiety present on the ligand agent to be coupled thereto. As an example, if the ligand agent comprises a reactive amino group, suitable reactive moieties for the amphiphilic molecule may be isothiocyanate groups (that will form a thiourea bond), reactive esters (to form an amide bond), aldehyde groups (for the formation of an imine bond to be reduced to an alkylamine bond), etc.; if the ligand agent comprises a reactive thiol group, suitable complementary reactive moieties for the amphiphilic molecule include haloacetyl derivatives or maleimides (to form a thioether bond); and if the ligand agent comprises a reactive carboxylic group, suitable reactive moieties for the amphiphilic molecule might be amines and hydrazides (to form amide or alkylamide bonds). The reactive moiety can be linked directly to a phospholipid molecule or, preferably, to a spacer covalently linked to the phospholipid, to form the desired precursor of the ligand compound. Preferred precursors of ligand compounds include phospholipids, preferably phosphatidylethanolamines (PE) such as DMPE, DPPE, DSPE or DOPE. Particularly preferred are phospholipids, in particular PE, bearing a spacer moiety, preferably a hydrophilic polymer, e.g a polyethyleneglycol. DPPE-PEG1000, DPPE-PEG2000, DPPE-PEG3400, DSPE-PEG1000, DSPE-PEG2000 and DSPE-PEG3400 (bearing a suitable reactive moiety as above illustrated), are particularly preferred.

As explained more in detail in the following, the process of the invention can be used to prepare a gas-filled microbubble comprising a moiety of an affinity binding pair (e.g. streptavidin), thus obtaining a precursor of a targeted gas-filled microbubble. A targeting ligand (e.g. an antibody), bearing a complementary moiety of said affinity binding pair (e.g. biotin), can then be associated with said amphiphilic compound, by means of the affinity binding pair interaction, to obtain the desired targeted gas-filled microbubble. Preferably said affinity binding is effected at the end of the preparation process, in a aqueous suspension of said precursor of said targeted gas-filled microbubble obtained by reconstituting the lyophilized product.

Examples of suitable specific targets to which the microbubbles of the invention can be directed are, for instance, fibrin, the αᵥβ₃ receptor or the GPIIbIIIa receptor on activated platelets. Fibrin and platelets are in fact generally present in “thrombi”, i.e. coagula which may form in the blood stream and cause a vascular obstruction. Suitable binding peptides are disclosed, for instance, in the above cited U.S. Pat. No. 6,139,819. Further binding peptides specific for fibrin-targeting are disclosed, for instance, in International patent application WO 02/055544, which is herein incorporated by reference.

Other examples of important targets include receptors in vulnerable plaques and tumor specific receptors, such as kinase insert domain region (KDR) and VEGF (vascular endothelial growth factor)/KDR complex. Binding peptides suitable for KDR or VEGF/KDR complex are disclosed, for instance, in International Patent application WO 03/74005 and WO 03/084574, both herein incorporated by reference.

Process

The emulsifying step a) of the process of the present invention can be carried out by submitting the aqueous medium and the core solvent in the presence of at least one phospholipid to any appropriate emulsion-generating technique known in the art, such as, for instance, sonication, shaking, high pressure homogenization, micromixing, membrane emulsification, high speed stirring or high shear mixing, e.g. using a rotor-stator homogenizer. For instance, a rotor-stator homogenizer is employed, such as Polytron™ PT3000. The agitation speed of the rotor-stator homogenizer can be selected depending from the components of the emulsion, the volume of the emulsion and of the diameter of the vessel containing the emulsion and the desired final diameter of the microdroplets of solvent in the emulsion. In general, it has been observed that, when using a rotor-stator homogenizer having a probe of about 3 cm diameter immersed in a 50-80 ml mixture contained in 3.5-5 cm diameter beaker, an agitation speed of about 8000 rpm is typically sufficient to obtain microdroplets having a mean numerical diameter sufficiently reduced to result, after lyophilization and reconstitution of the lyophilized matrix, in gas-filled microbubbles having a diameter of less than about 1.8 μm. By increasing the agitation speed at about 12000 rpm, it is in general possible to obtain gas-filled microbubbles having a number mean diameter of less than about 1.5 μm, while with an agitation speed of about 14000-15000 rpm, gas-filled microbubbles having a number mean diameter of about 1.0 μm or less can generally be obtained. In general it has been observed that by increasing the agitation speed above about 18000 rpm, slight further reduction of microbubbles size is obtained.

Alternatively, a micromixing technique can also be employed for emulsifying the mixture. As known, a micromixer typically contain at least two inlets and at least one outlet. The organic solvent is thus introduced into the mixer through a first inlet (at a flow rate of e.g. 0.05-5 ml/min), while the aqueous phase is introduced through the second inlet (e.g. at a flow rate of 2-100 ml/min). The outlet of the micromixer is then connected to the vessel containing the aqueous, so that the aqueous phase drawn from said vessel at subsequent instants and introduced into the micromixer contains increasing amounts of emulsified solvent. When the whole volume of solvent has been added, the emulsion from the container can be kept under recirculation through the micromixer for a further predetermined period of time, e.g. 5-120 minutes, to allow completion of the emulsion.

Depending on the emulsion technique, the organic solvent can be introduced gradually during the emulsification step or at once before starting the emulsification step. Alternatively the aqueous medium may be gradually added to the water immiscible solvent during the emulsification step or at once before starting the emulsification step. The phospholipid can be either dispersed in the aqueous medium or in the organic solvent, before admixing the two, or it may be separately added the aqueous-organic mixture before or during the emulsification step. Preferably, the phospholipid is dispersed in the organic solvent (preferably cyclooctane). As previously mentioned, the lyprotective agent is preferably dispersed in the aqueous phase, to which the organic phase is then added.

The emulsification of step a) is conveniently carried out at room temperature, e.g. at a temperature of 22° C.±5° C., or at higher temperatures, for instance 50° C.-60° C. (e.g. in the core solvents with high boiling points) or at lower temperature, for instance 0° C.-10° C. (e.g. in the case of core solvents with boiling points close to room temperature). The temperature is preferably kept below the boiling temperature of the organic solvent, preferably at least 5° C. below said temperature, more preferably at least 10° C. below. As prolonged exposure of the mixture at high temperatures (e.g. 90° C. or more) may cause possible degradations of phospholipids, with consequent formation of of the respective lyso-derivatives, it is in general preferred to avoid such prolonged heating at high temperatures.

If necessary, the aqueous medium containing the phospholipids can be subjected to controlled heating, in order to facilitate the dispersion thereof. For instance, the phospholipid containing aqueous suspension can be heated at about 60-70° C. for about 15 minutes and then allowed to cool at the temperature at which the emulsion step is then carried out.

As previously mentioned, additional amphiphilic materials, such as those previously listed, can also be introduced into the emulsifying mixture containing the phospholipid. The amount of said additional amphiphilic compounds is preferably not higher than about 50% by weight with respect to the total weight of amphiphilic material, more preferably not higher than 20% by weight, down to an amount of e.g. about 0.1%.

The obtained emulsion typically comprises micro-droplets of solvent in the aqueous medium, with the amphiphilic compounds (including phospholipids) disposed at the water-solvent interface.

The aqueous medium may, if desired, further contain one or more excipients.

As used herein, the term “excipient” refers to any additive useful in the present invention, such as those additives employed to increase the stability of the emulsion or of the lyophilisate intermediate and/or to provide for pharmaceutically acceptable and stable final compositions.

Exemplary excipients in this regard are, for instance, viscosity enhancers and/or solubility aids for the phospholipids.

Viscosity enhancers and solubility aids that may suitably be employed are for example mono- or polysaccharides, such as glucose, lactose, saccharose, and dextrans, aliphatic alcohols, such as isopropyl alcohol and butyl alcohol, polyols such as glycerol, 1,2-propanediol, and the like agents. In general however we have found that it is unnecessary to incorporate additives such as viscosity enhancers, which are commonly employed in many existing contrast agent formulations, into the contrast agents of the present invention. This is a further advantage of the present invention as the number of components administered to the body of a subject is kept to a minimum and the viscosity of the contrast agents is maintained as low as possible.

As mentioned before, the Applicant has found substantially unnecessary, to add water-insoluble structure-builders, such as cholesterol, to the emulsifying mixture. As a matter of fact, it has been observed that an amount of 0.05% (w/w with respect to the total weight of the emulsifying mixture) of cholesterol dramatically reduces the conversion yield from microdroplets into gas-filled microvesicles, further resulting in a broad-dispersion of the vesicles' size. The amount of water-insoluble compounds in the emulsifying mixture, particularly of those compounds not comprising one or two fatty acid residue in their structure, is thus preferably lower than 0.050%, more preferably lower than about 0.030% by weight with respect to the total weight of the emulsion.

According to step b) of the process of the invention, a precursor of said ligand agent is added to the formed emulsion. Preferably, an aqueous suspension of the desired precursor of the ligand agent is first prepared and then added to the formed emulsion, preferably under agitation and heating (preferably at less than 80° C., e.g. 40° C.-80° C., in particular 50-70° C.). As mentioned, the precursor of the ligand agent is an (amphiphilic) compound comprising a suitable reactive moiety, which can be reacted with a complementary reactive moiety contained in the ligand agent. For example, a micellar suspension of a PE-PEG (e.g. DPPE- or DSPE-PEG2000) containing a suitable reactive moiety (e.g. maleimide) can be prepared and then admixed with the formed emulsion. This step of the preparation method allows an effective incorporation of the precursor (e.g. PE-PEG-maleimide) of the ligand agent into the layer of amphiphilic material surrounding the microdroplets of solvent. The precursor of the ligand agent is preferably added in a molar amount of from about 0.1% to about 10% with respect to the amphiphilic material employed in the emulsion of step a. More preferably, said molar amount is from about 1% to about 5% and even more preferably of a from about 2% to 3%.

If desired, additional amphiphilic compounds or polymeric surfactants may also be introduced together with the precursor during this step. Examples of amphiphilic compounds which can conveniently be introduced after the preparation of the initial emulsion are, for instance, PEG-modified phospholipids, in particular PEG-modified phosphatidylethanolamines, such as DMPE-PEG750, DMPE-PEG1000, DMPE-PEG2000, DMPE-PEG3000, DMPE-PEG4000, DMPE-PEG5000, DPPE-PEG750, DPPE-PEG1000, DPPE-PEG2000, DPPE-PEG3000, DPPE-PEG4000, DPPE-PEG5000, DSPE-PEG750, DSPE-PEG1000, DSPE-PEG2000, DSPE-PEG3000, DSPE-PEG4000, DSPE-PEG5000, DAPE-PEG750, DAPE-PEG1000, DAPE-PEG2000, DAPE-PEG3000, DAPE-PEG4000 or DAPE-PEG5000. Examples of polymeric surfactants which can be conveniently added after formation of the emulsion are, for instance, ethyleneoxide-propylenoxide block copolymers, such as Pluronic F68, Pluronic F108, Pluronic F-127 (Sigma Aldrich, Missouri, USA); Polyoxyethylated alkyl ethers such as Brij® 78 (Sigma Aldrich, Missouri, USA); Polyoxyethylene fatty acid esters such as Myrj® 53 or Myrj® 59 (Sigma Aldrich, Missouri, USA); Polyoxyethylenesorbitan fatty acid ester such as Tween® 60 (Sigma Aldrich, Missouri, USA); or Polyethylene glycol tert-octylphenyl ether such as Triton® X-100 (Sigma Aldrich, Missouri, USA).

Optionally, the emulsion can be subjected to a controlled additional heating treatment after the admixing with the ligand agent. The additional heating of the emulsion is preferably performed into a sealed container. The heat treatment can vary from about 15 minutes to about 90 minutes, at temperatures comprised from about 60° C. to about 125° C., preferably from about 80° C. to about 120° C. In general, the higher the temperature, the shortest the time of the thermal treatment. During the heating, the emulsion can optionally be kept under agitation.

As observed by the Applicant, while this additional thermal treatment may result in a partial degradation of the phospholipids (e.g. with a content of about 5-20% w/w of lysolipids in the final product, when the emulsion is heated at about 100-120° C. for about 30 min) and/or of the of the precursor, it may nevertheless allow a substantial narrowing of the size distribution and an increase of the total number of microbubbles in the final suspension, independently from the working conditions of the initial emulsification step (e.g. type of organic solvent, emulsifying technique, optional washing steps, etc.).

According to step c) of the process of the invention, the precursor is converted into the ligand agent. Thus, a compound comprising said ligand agent and a suitable reactive moiety (complementary with the one on the precursor) is introduced in the emulsion, preferably in the form of an aqueous suspension, and reacted with the precursor (i.e. with the corresponding reactive moiety o the precursor) to form the desired ligand agent.

According to an embodiment of the invention, the ligand agent is a targeting ligand, such as those previously mentioned, preferably an antibody or fragments thereof. Antibodies include mono-, bi- and multi-specific antibodies, i.e. antibody molecules having affinity and/or specificity for one, two or more different antigens, respectively. Examples of suitable antibodies, and of their respective potential target, where available, are illustrated in the following table.

| Antibody | Target | Comment/area of use |
|---|---|---|
| Anti ICAM-1/CD54 | Intracellular Adhesion Molecule-1 | Endothelial cells activation |
| Anti ICAM-2 | Intracellular Adhesion Molecule-2 | as above |
| Anti CD62E | L-Selectin | as above |
| Anti CD62P | P-Selectin | as above |
| Anti CD31 | PECAM-1 | as above |
| Anti-TM/CD141 | Thrombomodulin | as above |
| Anti-VCAM-1/CD106 | vascular cell adhesion molecule-1 | as above |
| Anti CD105 | Endoglin | marker of angiogenic |
| | | endothelial cells |
| Anti Endocan | Endothelial cell specific | as above |
| | molecule-1 (ESM-1) | |
| Anti-KDR/Flk-1 | Vascular endothelium growth | as above |
| | factor Receptor-2 | |
| Anti-Flt-1 | Vascular endothelium growth | as above |
| | factor Receptor-1 | |
| Anti-Nucleolin | | as above |
| Anti-TEM1 | Tumor endothelial marker | as above |
| | 1/endosialin | |
| Anti-TEM5 | Tumor endothelial marker 5 | as above |
| Anti-TEM7 | Tumor endothelial marker 7 | as above |
| Anti-TEM8 | Tumor endothelial marker 8 | as above |
| Anti-TF | Tissue Factor | as above |
| Anti PSMA | Prostate Specific Membrane | as above |
| | Antigen | |
| Anti-CXCR4 | | marker of angiogenic |
| | | endothelial cells |
| Anti-NRP1 | Neuropilin-1 | marker of angiogenic |
| | | endothelial cells |
| Integrins, | | endothelial cell marker |
| e.g. VLA-1, | | |
| VLA-2, VLA- | | |
| 3, VLA-4, | | |
| VLA-5, VLA- | | |
| 6, α7 β1, αv β3, | | |
| LFA-1, Mac- | | |
| 1, CD41a, etc. | | |
| Anti-VE-cadherin | | as above |
| (CD144) | | |
| Anti-vWF | von Willerbrand factor | as above |
| Anti CD34 | CD34/gp 105-120 | as above |

Examples of fragments of antibodies are, for instance, Fab fragments (papain digested whole antibody), F(ab′)2 fragment (pepsin digested whole antibody), single chain variable fragment (scFv), nanobodies (the single variable region of naturally occurring heavy chain antibodies). Preferred fragments are Fab fragments.

According to an alternative embodiment, said ligand agent is a moiety of an affinity binding pair, such as those previously described, preferably streptavidin.

The amount of ligand agent added to the emulsion will depend from the type of ligand agent employed. In general, the compound containing the targeting ligand is added in molar defect with respect to the precursor in the emulsion. Preferably the molar ratio between the compound comprising the ligand agent and the precursor in the emulsion is of from about 1:2 to about 1:10000, more preferably of from about 1:5 to 1:1000 and even more preferably from about 1:10 to about 1:100. Said molar amounts generally result in corresponding concentrations of ligand agent on the surface of the gas-filled microbubbles of from about 10 to about 10⁵ molecules of ligand agent per μm² of the surface of microbubbles. Preferably, said concentration is of from about 100 to about 10000 molecules of ligand agent per μm² and more of from about 1000 to about 8000 molecules of ligand agent per μm². The obtained emulsion can then be directly subjected to lyophilization, typically without the need of further washing steps.

According to an alternative embodiment of the invention, the precursor of the ligand agent can be converted into the ligand agent before adding said precursor to the aqueous-organic emulsion, so that the ligand agent is directly added to the emulsion. For instance, if an aqueous micellar suspension of the precursor of the ligand agent is prepared (e.g. PE-PEG-mal), said precursor may be converted into the ligand agent (e.g. PE-PEG-antibody) in said micellar suspension, by reacting a suitably functionalized ligand agent (e.g. antibody-SH) with said precursor.

Lyophilization of the final emulsion may be carried out by initially freezing the emulsion and thereafter lyophilizing the frozen emulsion, by per se generally known methods and devices. Since the dried, lyophilized, product will normally be reconstituted by addition of a carrier liquid prior to administration, the emulsion may advantageously be filled into sealable vials prior to lyophilization so as to give vials each containing an appropriate amount, e.g. a single dosage unit, of lyophilized dried product for reconstitution into an injectable form. By lyophilizing the emulsion in individual vials rather than in bulk, handling of the delicate honeycomb-like structure of the lyophilized product and the risk of at least partially degrading this structure are avoided.

Following lyophilization, the vacuum can be removed in the lyophilizer by introducing the desired gas to form the microbubbles in the final formulation of the contrast agent. This will allow to fill the headspace of the vials with the desired gas and then seal the vials with an appropriate closure. Alternatively, the vial can be kept under vacuum and sealed, while the gas is added at a later stage, e.g. just before administration, for instance when the gas is a radioactive or a hyperpolarized gas.

The so obtained lyophilized product in the presence of the suitable gas can thus be stably stored for several months before being reconstituted by dissolving it into an aqueous carrier liquid, to obtain a suspension of gas-filled microbubbles.

Any biocompatible gas, gas precursor or mixture thereof may be employed to fill the above microvesicles, the gas being selected depending on the chosen modality.

The gas may comprise, for example, air; nitrogen; oxygen; carbon dioxide; hydrogen; nitrous oxide; a noble or inert gas such as helium, argon, xenon or krypton; a radioactive gas such as Xe¹³³ or Kr⁸¹; a hyperpolarized noble gas such as hyperpolarized helium, hyperpolarized xenon or hyperpolarized neon; a low molecular weight hydrocarbon (e.g. containing up to 7 carbon atoms), for example an alkane such as methane, ethane, propane, butane, isobutane, pentane or isopentane, a cycloalkane such as cyclobutane or cyclopentane, an alkene such as propene, butene or isobutene, or an alkyne such as acetylene; an ether; a ketone; an ester; halogenated gases, preferably fluorinated gases, such as or halogenated, fluorinated or perfluorinated low molecular weight hydrocarbons (e.g. containing up to 7 carbon atoms); or a mixture of any of the foregoing. Where a halogenated hydrocarbon is used, preferably at least some, more preferably all, of the halogen atoms in said compound are fluorine atoms.

Fluorinated gases are preferred, in particular perfluorinated gases, especially in the field of ultrasound imaging. Fluorinated gases include materials which contain at least one fluorine atom such as, for instance fluorinated hydrocarbons (organic compounds containing one or more carbon atoms and fluorine); sulfur hexafluoride; fluorinated, preferably perfluorinated, ketones such as perfluoroacetone; and fluorinated, preferably perfluorinated, ethers such as perfluorodiethyl ether. Preferred compounds are perfluorinated gases, such as SF₆ or perfluorocarbons (perfluorinated hydrocarbons), i.e. hydrocarbons where all the hydrogen atoms are replaced by fluorine atoms, which are known to form particularly stable microbubble suspensions, as disclosed, for instance, in EP 0554 213, which is herein incorporated by reference.

The term perfluorocarbon includes saturated, unsaturated, and cyclic perfluorocarbons. Examples of biocompatible, physiologically acceptable perfluorocarbons are: perfluoroalkanes, such as perfluoromethane, perfluoroethane, perfluoropropanes, perfluorobutanes (e.g. perfluoro-n-butane, optionally in admixture with other isomers such as perfluoro-isobutane), perfluoropentanes, perfluorohexanes or perfluoroheptanes; perfluoroalkenes, such as perfluoropropene, perfluorobutenes (e.g. perfluorobut-2ene) or perfluorobutadiene; perfluoroalkynes (e.g. perfluorobut-2-yne); and perfluorocycloalkanes (e.g. perfluorocyclobutane, perfluoromethylcyclobutane, perfluorodimethylcyclobutanes, perfluorotrimethylcyclobutanes, perfluorocyclopentane, perfluoromethylcyclopentane, perfluorodimethylcyclopentanes, perfluorocyclohexane, perfluoromethylcyclohexane and perfluorocycloheptane). Preferred saturated perfluorocarbons have the formula CₙFₙ₊₂, where n is from 1 to 12, preferably from 2 to 10, most preferably from 3 to 8 and even more preferably from 3 to 6. Suitable perfluorocarbons include, for example, CF₄, C₂F₆, C₃F₈, C₄F₈, C₄F₁₀, C₅F₁₂, C₆F₁₂, C₆F₁₄, C₇F₁₄, C₇F₁₆, C₈F₁₈, and C₉F₂₀.

Particularly preferred gases are SF₆ or perfluorocarbons selected from CF₄, C₂F₆, C₃F₈, C₄F₈, C₄F₁₀ or mixtures thereof; SF₆, C₃F₈ or C₄F₁₀ are particularly preferred.

It may also be advantageous to use a mixture of any of the above gases in any ratio. For instance, the mixture may comprise a conventional gas, such as nitrogen, air or carbon dioxide and a gas forming a stable microbubble suspension, such as sulfur hexafluoride or a perfluorocarbon as indicated above. Examples of suitable gas mixtures can be found, for instance, in WO 94/09829, which is herein incorporated by reference. The following combinations are particularly preferred: a mixture of gases (A) and (B) in which the gas (B) is a fluorinated gas, preferably selected from SF₆, CF₄, C₂F₆, C₃F₆, C₃F₈, C₄F₆, C₄F₈, C₄F₁₀, C₅F₁₀, C₅F₁₂ or mixtures thereof, more preferably SF₆, C₃F₈, or C₄F10, and (A) is selected from air, oxygen, nitrogen, carbon dioxide or mixtures thereof. The amount of gas (B) can represent from about 0.5% to about 95% v/v of the total mixture, preferably from about 5% to 80%.

In some instances it may be desirable to include a precursor to a gaseous substance (i.e. a material that is capable of being converted to a gas in vivo). Preferably the gaseous precursor and the gas derived therefrom are physiologically acceptable. The gaseous precursor may be pH-activated, photo-activated, temperature activated, etc. For example, certain perfluorocarbons may be used as temperature activated gaseous precursors. These perfluorocarbons, such as perfluoropentane or perfluorohexane, have a liquid/gas phase transition temperature above room temperature (or the temperature at which the agents are produced and/or stored) but below body temperature; thus, they undergo a liquid/gas phase transition and are converted to a gas within the human body. Furthermore, he term “gas” as used herein includes mixtures in vapor form at the normal human body temperature of 37° C. Compounds which at the temperature of 37° C. are liquid may thus also be used in limited amounts in admixture with other gaseous compounds, to obtain a mixture which is in a vapor phase at 37° C.

For ultrasonic echography, the biocompatible gas or gas mixture is preferably selected from air, nitrogen, carbon dioxide, helium, krypton, xenon, argon, methane, halogenated hydrocarbons (including fluorinated gases such as perfluorocarbons and sulfur hexafluoride) or mixtures thereof. Advantageously, perfluorocarbons (in particular C₄F₁₀ or C₃F₈) or SF₆ can be used, optionally in admixture with air or nitrogen.

For the use in MRI the microbubbles will preferably contain a hyperpolarized noble gas such as hyperpolarized neon, hyperpolarized helium, hyperpolarized xenon, or mixtures thereof, optionally in admixture with air, CO₂, oxygen, nitrogen, helium, xenon, or any of the halogenated hydrocarbons as defined above.

For use in scintigraphy, the microbubbles according to the invention will preferably contain radioactive gases such as Xe¹³³ or Kr⁸¹ or mixtures thereof, optionally in admixture with air, CO₂, oxygen, nitrogen, helium, kripton or any of the halogenated hydrocarbons as defined above.

The lyophilized composition in contact with the gas can then be very easily reconstituted by the addition of an appropriate sterile aqueous injectable and physiologically acceptable carrier liquid such as sterile pyrogen-free water for injection, an aqueous solution such as saline (which may advantageously be balanced so that the final product for injection is not hypotonic), or an aqueous solution of one or more tonicity-adjusting substances such as salts (e.g. of plasma cations with physiologically tolerable counterions), or sugars, sugar alcohols, glycols and other non-ionic polyol materials (e.g. glucose, sucrose, sorbitol, mannitol, glycerol, polyethylene glycols, propylene glycols and the like), requiring only minimal agitation such as may, for example, be provided by gentle hand-shaking.

Where the dried product is contained in a vial, this is conveniently sealed with a septum through which the carrier liquid may be injected using an optionally pre-filled syringe; alternatively the dried product and carrier liquid may be supplied together in a dual chamber device such as a dual chamber syringe. It may be advantageous to mix or gently shake the product following reconstitution. However, as noted above, in the stabilized contrast agents according to the invention the size of the gas microbubbles may be substantially independent of the amount of agitation energy applied to the reconstituted dried product. Accordingly no more than gentle hand-shaking may be required to give reproducible products with consistent microbubble size.

The microbubble suspensions generated upon reconstitution in water or an aqueous solution may be stable for at least 12 hours, thus permitting considerable flexibility as to when the dried product is reconstituted prior to injection.

Unless it contains a hyperpolarized gas, known to require special storage conditions, the lyophilised residue may be stored and transported without need of temperature control of its environment and in particular it may be supplied to hospitals and physicians for on site formulation into a ready-to-use administrable suspension without requiring such users to have special storage facilities.

Preferably in such a case it can be supplied in the form of a two component kit.

Said two component kit can include two separate containers or a dual-chamber container. In the former case preferably the container is a conventional septum-sealed vial, wherein the vial containing the lyophilized residue of step b) is sealed with a septum through which the carrier liquid may be injected using an optionally prefilled syringe. In such a case the syringe used as the container of the second component is also used then for injecting the contrast agent. In the latter case, preferably the dual-chamber container is a dual-chamber syringe and once the lyophilisate has been reconstituted and then suitably mixed or gently shaken, the container can be used directly for injecting the contrast agent. In both cases means for directing or permitting application of sufficient bubble forming energy into the contents of the container are provided. However, as noted above, in the stabilised contrast agents according to the invention the size of the gas microbubbles is substantially independent of the amount of agitation energy applied to the reconstituted dried product. Accordingly no more than gentle hand shaking is generally required to give reproducible products with consistent microbubble size.

It can be appreciated by one ordinary skilled in the art that other two-chamber reconstitution systems capable of combining the dried powder with the aqueous solution in a sterile manner are also within the scope of the present invention. In such systems, it is particularly advantageous if the aqueous phase can be interposed between the water-insoluble gas and the environment, to increase shelf life of the product. Where a material necessary for forming the contrast agent is not already present in the container (e.g. a targeting ligand to be linked to the phospholipid during reconstitution), it can be packaged with the other components of the kit, preferably in a form or container adapted to facilitate ready combination with the other components of the kit.

No specific containers, vial or connection systems are required; the present invention may use conventional containers, vials and adapters. The only requirement is a good seal between the stopper and the container. The quality of the seal, therefore, becomes a matter of primary concern; any degradation of seal integrity could allow undesirables substances to enter the vial. In addition to assuring sterility, vacuum retention is essential for products stoppered at ambient or reduced pressures to assure safe and proper reconstitution. As to the stopper, it may be a compound or multicomponent formulation based on an elastomer, such as poly(isobutylene) or butyl rubber.

When the ligand agent included in the gas-filled microbubbles is a targeting ligand, the reconstituted suspension can be used in any contrast specific imaging method as described in detail hereinafter.

When the ligand agent included in the gas-filled microbubbles is a moiety of an affinity binding pair (e.g. streptavidin), the microbubbles in the reconstituted suspension can then be associated with a targeting ligand comprising a complementary moiety of said binding pair (e.g. a byotinilated antibody). Said association can easily be effected by admixing an aqueous suspension of the functionalized targeting ligand with the reconstituted suspension of microbubbles (e.g. in the reconstitution vial), preferably under agitation. Agitation can be manual mechanical agitation of the vial, for a period of time of about 1 to 20 minutes, preferably for about 10 minutes. Preferably, the ratio between the molar amount of the targeting ligand containing a moiety of a binding pair (e.g. a biotinylated antibody) and the molar amount of the precursor containing the complementary moiety (e.g. streptavidin) in the suspension of microbubbles is from about 1:2 to about 20:1, more preferably from about 1:1 to about 10:1, and even more preferably from about 2:1 to about 8:1. The so obtained targeted microbubbles can then be used in any contrast specific imaging method as described in detail hereinafter.

By suitably selecting the components of the mixture and in particular the amount of agitation energy applied during the emulsion of the aqueous-organic mixture, it is possible to obtain gas-filled microbubbles with the desired numerical mean diameter and size distribution.

According to an embodiment, by exploiting the process according to the present invention it is possible to obtain contrast agents comprising phospholipid-stabilized small-sized gas microbubbles characterized by having relatively small mean dimensions and a particularly useful narrow and controlled size distribution. For instance, the process of the invention allows to obtain gas-filled microbubbles compositions where at least 10% of the total volume of the gas in the microbubbles is contained in microbubbles with a diameter of 1.5 μm or less. Alternatively, the process of the present invention also allows to prepare microbubbles having a mean diameter in number (D_{N}) of less than 1.70 μm and a median diameter in volume (D_{V50}) such that the D_{V50}/D_{N} ratio is of about 2.30 or lower.

The concentration of microbubbles in the reconstituted suspension is in general of at least 1×10⁸ particles per milliliter, preferably of at least 1×10⁹ particles per milliliter.

The above values of gas amount, D_{V50}, D_{N} and number of microbubbles are referred to a measurement made by using a Coulter Counter Mark II apparatus fitted with a 30 μm aperture, with a measuring range of 0.7 to 20 μm.

The contrast agents obtainable by the process of the present invention may be used in a variety of diagnostic imaging techniques, including in particular ultrasound and Magnetic Resonance. Possible other diagnostic imaging applications include scintigraphy, light imaging, and X-ray imaging,including X-ray phase contrast imaging.

Their use in diagnostic ultrasound imaging and in MR imaging, e.g. as susceptibility contrast agents and as hyperpolarized gas bubbles, are particularly preferred. Ultrasound imaging techniques that can be used include known techniques, such as color Doppler, power Doppler, Doppler amplitude, stimulated acoustic imaging, and two- or three-dimensional imaging techniques. Imaging may be done in harmonic (resonant frequency) or fundamental modes, with the second harmonic preferred.

In ultrasound applications, the contrast agents formed by phospholipid stabilized microbubbles may, for example, be administered in doses such that the amount of phospholipid injected is in the range 0.1 to 200 μg/kg body weight, preferably from about 0.1 to 30 μg/kg.

For ultrasound applications such as echocardiography, in order to permit free passage through the pulmonary system and to achieve resonance with the preferred imaging frequencies of about 0.1-15 MHz, microbubbles having an average size of 0.1-10 μm, e.g. 0.5-7 μm are generally employed. As described above, contrast agents according to the invention may be produced with a very narrow size distribution for the microbubble dispersion within the range preferred for echocardiography, thereby greatly enhancing their echogenicity as well as their safety in vivo.

According to alternative embodiments, the ultrasound contrast agents obtained according to the process of the invention, in particular streptavidin-containing microbubbles associated to biotinylated antibodies, can be used in laboratory diagnostic imaging on animals, e.g. to follow the results of a therapeutic treatment.

The ultrasound contrast agents obtained according to the process of the invention may further be used in a variety of therapeutic imaging methods. The term therapeutic imaging includes within its meaning any method for the treatment of a disease in a patient which comprises the use of a contrast imaging agent (e.g. for the delivery of a therapeutic agent to a selected receptor or tissue), and which is capable of exerting or is responsible to exert a biological effect in vitro and/or in vivo. Therapeutic imaging may advantageously be associated with the controlled localized destruction of the gas-filled microbubbles, e.g. by means of an ultrasound burst at high acoustic pressure (typically higher than the one generally employed in non-destructive diagnostic imaging methods). This controlled destruction may be used, for instance, for the treatment of blood clots (a technique also known as sonothrombolysis), optionally in combination with the localized release of a suitable therapeutic agent. Alternatively, said therapeutic imaging may include the delivery of a therapeutic agent into cells, as a result of a transient membrane permeabilization at the cellular level induced by the localized burst of the microvesicles. This technique can be used, for instance, for an effective delivery of genetic material into the cells; optionally, a drug can be locally delivered in combination with genetic material, thus allowing a combined pharmaceutical/genetic therapy of the patient (e.g. in case of tumor treatment).

The term “therapeutic agent” includes within its meaning any substance, composition or particle which may be used in any therapeutic application, such as in methods for the treatment of a disease in a patient, as well as any substance which is capable of exerting or responsible to exert a biological effect in vitro and/or in vivo. Therapeutic agents thus include any compound or material capable of being used in the treatment (including diagnosis, prevention, alleviation, pain relief or cure) of any pathological status in a patient (including malady, affliction, disease lesion or injury). Examples of therapeutic agents are drugs, pharmaceuticals, bioactive agents, cytotoxic agents, chemotherapy agents, radiotherapeutic agents, proteins, natural or synthetic peptides, including oligopeptides and polypeptides, vitamins, steroids and genetic material, including nucleosides, nucleotides, oligonucleotides, polynucleotides and plasmids. In a preferred embodiment the therapeutic agent is a drig useful in the treatment of cancer, thromobotic disorders or angiogenic disorders.

The following non-limitative examples may further illustrate the invention.

### EXAMPLES

The following materials have been employed in the following examples.

Phospholipids:

DPPS Dipalmitoylphosphatidylserine (Genzyme) IUPAC: 1,2-Dipalmitoyl-sn-glycero-3-phosphoserine
DPPG Dipalmitoylphosphatidylglycerol sodium salt (Genzyme) IUPAC: 1,2-Dipalmitoyl-sn-glycero-3-[phospho-rac-(1-glycerol)]
DSPA Distearoyl phosphatidic acid sodium salt (Genzyme) IUPAC: 1,2-Distearoyl-sn-glycero-3-phosphate
DSPG Distearoylphosphatidylglycerol sodium salt (Genzyme) IUPAC: 1,2-Distearoyl-sn-glycero-3-phospho-rac-(1-glycerol))
DPPS Dipalmitoylphosphatidylserine (Genzyme) IUPAC: 1,2-Dipalmitoyl-sn-glycero-3-phosphocholine
DPPG Dipalmitoylphosphatidylglycerol sodium salt (Genzyme) IUPAC: 1,2-Dipalmitoyl-sn-glycero-3-[phospho-rac-(1-glycerol)]
DSPA Distearoyl phosphatidic acid sodium salt (Genzyme) IUPAC: 1,2-Distearoyl-sn-glycero-3-phosphate
DSPG Distearoylphosphatidylglycerol sodium salt (Genzyme) IUPAC: 1,2-Distearoyl-sn-glycero-3-phosphoserine)
DSPC Distearoylphosphatidylcholine (Genzyme) IUPAC: 1,2-Distearoyl-sn-glycero-3-phosphocholine
DSEPC Distearoylethylphosphatidylcholine (Avanti Polar Lipids) IUPAC: 1,2-Distearoyl-sn-glycero-3-Ethylphosphocholine
DAPC Diarachidoylphosphatidylcholine (Avanti polar Lipids) IUPAC: 1,2-Diarachidoyl-sn-glycero-3-phosphocholine
DSTAP 1,2-Distearoyl-3-trimethylammonium-propane chloride (Avanti Polar Lipids)
DSPE-PEG2000 Distearoylphosphatidylethanolamine modified with PEG2000, sodium salt (Nektar Therapeutics)
DSPE-PEG5000 Distearoylphosphatidylethanolamine modified with PEG5000, sodium salt (Nektar Therapeutics)
DSPE-PEG2000-mal Distearoylphosphatidylethanolamine modified with PEG2000-maleimide (Avanti Polar lipids)
SATA N-Succinimidyl-S-acetylthioacetate (Pierce)
RGD-4C H-Ala-Cys-Asp-Cys-Arg-Gly-Asp-Cys-Phe-Cys-Gly-NH₂ (AnaSpec Inc.)
   Solvents:
Perfluoro-n-hexane (C₆F₁₄), by Fluka
perfluoromethylcyclohexane (CF₃-cyclo-C₆F₁₁), by Fluka
perfluoro-n-heptane (C₇F₁₆), by Fluka
perfluoro-n-nonane (C₉F₂₀), by Aldrich
perfluorodecalin, by Aldrich
Cyclohexane, by Fluka
Cyclooctane, by Fluka
n-Decane, by Fluka
n-Octane, by Fluka
meta xylene, by Fluka
Diisopropyl cetone, by Fluka
CCl₄, by Fluka
   Lyoprotectants:
Mannose, by Fluka
Glucose, by Fluka
Sorbitol, by Fluka
Mannitol, by Fluka
Maltose, by Fluka
Dextran 6000, by Fluka
Dextran 15000, by Fluka
Dextran 40000, by Fluka
Inulin, by Fluka
   Characterization of Microdroplets and Microbubbles.

The size distribution of the emulsions microdroplets has been determined:
a) by means of a Coulter counter (Counter Mark II apparatus fitted with a 30 μm aperture with a measuring range of 0.7 to 20 μm), when the emulsion has been submitted to a washing step; 10 μl of emulsion were diluted in 100 ml of saline at room temperature and allowed to equilibrate for 3 minutes prior to measurement;
b) by means of a laser light scattering particle sizer (Malvern Mastersizer, dilution 200×, focal length 45 mm, standard presentation), if the emulsion has not been subjected to a washing step.

The size distributions, volume concentrations and number of the microbubbles (after lyophilisation and reconstitution with an aqueous phase) were determined by using a Coulter Counter Mark II apparatus fitted with a 30 μm aperture with a measuring range of 0.7 to 20 μm. 50 μl of microbubble samples were diluted in 100 ml of saline at room temperature and allowed to equilibrate for 3 minutes prior to measurement.

The amounts of phospholipids in the final preparations (emulsion of microbubbles suspension) were determined by HPLC-MS analysis, with the following set up: Agilent 1100 LC chromatograph, Minn. CC 125/2 mm-5 C8 column from Maherey Nagel, Agilent MSD G1946D detector.

Lyophilization

The lyophilization methodology and apparatus, where not otherwise specified, were as follows. The emulsion (optionally after the washing step, if present) is first frozen at −45° C. for 5 minutes and then freeze-dried (lyophilized) at room temperature at a pressure of 0.2 mbar, by using a Christ-Alpha 2-4 freeze-drier.

### Example 1

### Preparations 1a-1n

10 mg of DPPS are added to about 10 ml of an 10% (w/w) mannitol aqueous solution; the suspension is heated at 65° C. for 15 minutes and then cooled at room temperature (22° C.). Perfluoroheptane (8% v/v) is added to this aqueous phase and emulsified in a beaker of about 4 cm diameter by using a high speed homogenizer (Polytron T3000, probe diameter of 3 cm) for 1 minute at the speed indicated in table 1. The resulting median diameter in volume (Dᵥ₅₀) and a mean diameter in number (D_{N}) of microdroplets of the emulsion are shown in table 1. The emulsion is then centrifuged (800-1200 rpm for 10 minutes, Sigma centrifuge 3K10) to eliminate the excess of the phospholipid and the separated pellets (microdroplets) were recovered and re-suspended in the same initial volume of a 10% mannitol aqueous solution.

The washed emulsion is then collected into a 100 ml balloon for lyophilization, frozen and then freeze-dried according to the above standard procedure. The lyophilized is then exposed to an atmosphere containing 35% of perfluoro-n-butane and 65% of nitrogen and then dispersed in a volume of water twice than the initial one by gentle hand shaking. The microbubble suspension obtained after reconstitution with distilled water is analyzed using a Coulter counter. The concentration of microbubbles in the obtained suspensions was of about 1×10⁹ particles per ml. The respective microbubbles median diameter in volume (Dᵥ₅₀), mean diameter in volume (D_{V}), mean diameter in number (D_{N}) an the amount of microbubbles with diameter larger than 3 μm (percentage over the total number of microbubbles) are given in table 1. When more than one example has been performed at the same agitation speed, the values indicated in table 1 are referred to the mean calculated value of each parameter.

TABLE 1

| | | | | Gas-filled microbubbles | | | | | |
|---|---|---|---|---|---|---|---|---|---|
| | EMULSION | | | | | | | | <1.5 |
| | Agita- | | | | | | | | μm |
| | tion | D_{V50} | D_{N} | D_{V50} | D_{V} | D_{N} | D_{V50}/ | >3 μm | vol. |
| Ex. | (rpm) | (μm) | (μm) | (μm) | (μm) | (μm) | D_{N} | part. % | % |
| 1a | 8000 | 4.58 | 1.77 | 2.92 | 3.33 | 1.51 | 1.93 | 5.44 | 10.9 |
| 1b | 9000 | 4.66 | 1.94 | 3.19 | 3.45 | 1.53 | 2.08 | 6.61 | 10.0 |
| 1c | 10000 | 3.04 | 1.74 | 2.16 | 2.53 | 1.33 | 1.62 | 1.88 | 22.6 |
| 1d | 11000 | 3.05 | 1.80 | 2.17 | 3.33 | 1.32 | 1.65 | 1.55 | 23.7 |
| 1e | 12000 | 2.84 | 1.69 | 1.86 | 2.17 | 1.24 | 1.50 | 0.93 | 33.1 |
| 1f | 12500 | 2.79 | 1.68 | 1.75 | 2.05 | 1.22 | 1.44 | 0.65 | 35.7 |
| 1g | 14000 | 2.20 | 1.52 | 1.39 | 2.45 | 1.08 | 1.29 | 0.23 | 58.0 |
| 1h | 14500 | 2.00 | 1.38 | 1.19 | 1.39 | 1.01 | 1.19 | 0.06 | 73.3 |
| 1i | 15000 | 1.88 | 1.39 | 1.22 | 2.20 | 1.01 | 1.21 | 0.06 | 70.3 |
| 1j | 15500 | 2.19 | 1.48 | 1.24 | 1.46 | 1.02 | 1.22 | 0.11 | 68.7 |
| 1k | 16000 | 1.83 | 1.32 | 1.27 | 3.08 | 0.99 | 1.28 | 0.10 | 65.2 |
| 1l | 17000 | 1.40 | 1.12 | 0.91 | 1.03 | 0.87 | 1.05 | 0.01 | 95.7 |

### Example 2

### Preparations 2a-2j

The same procedure adopted for example 1 is followed, with the only difference that the phospholipid is a mixture of DPPS (20% w/w) and DSPC (80% w/w), the total amount of phospholipid remaining unchanged. The results are summarized in table 2.

TABLE 2

| | | | | Gas-filled microbubbles | | | | | |
|---|---|---|---|---|---|---|---|---|---|
| | EMULSION | | | | | | | | <1.5 |
| | Agita- | | | | | | | | μm |
| | tion | D_{V50} | D_{N} | D_{V50} | D_{V} | D_{N} | D_{V50}/ | >3 μm | vol. |
| Ex. | (rpm) | (μm) | (μm) | (μm) | (μm) | (μm) | D_{N} | part. % | % |
| 2a | 6000 | 8.75 | 3.07 | 7.55 | 9.05 | 2.27 | 3.33 | 21.81 | 1.2 |
| 2b | 10000 | 3.54 | 1.90 | 3.00 | 3.71 | 1.47 | 2.04 | 5.05 | 11.7 |
| 2c | 12000 | 3.04 | 1.83 | 2.45 | 3.73 | 1.32 | 1.85 | 2.15 | 19.8 |
| 2d | 12500 | 2.85 | 1.76 | 2.21 | 3.24 | 1.27 | 1.74 | 1.57 | 24.4 |
| 2e | 13000 | 2.98 | 1.83 | 2.25 | 3.04 | 1.28 | 1.76 | 1.76 | 23.5 |
| 2f | 13500 | 2.91 | 2.05 | 1.88 | 2.46 | 1.20 | 1.57 | 0.87 | 33.8 |
| 2g | 14000 | 2.45 | 1.67 | 1.82 | 2.66 | 1.16 | 1.57 | 0.57 | 36.5 |
| 2h | 14500 | 2.18 | 1.55 | 1.58 | 3.04 | 1.09 | 1.44 | 0.38 | 46.5 |
| 2i | 15000 | 1.94 | 1.42 | 1.34 | 1.96 | 1.04 | 1.28 | 0.31 | 61.5 |
| 2j | 16000 | 1.81 | 1.38 | 1.35 | 2.30 | 1.03 | 1.31 | 0.14 | 59.0 |

### Example 3

### Preparation 3a-3p

The same procedure adopted for examples 2 is followed, with the only difference that the DPPS/DSPC weight ratio is varied, as reported in table 3. The results are summarized in table 3.

TABLE 3

| | | EMULSION | | | Gas-filled microbubbles | | | | |
|---|---|---|---|---|---|---|---|---|---|
| | DPPS/DSPC | Agitation | | D_{N} | D_{V50} | D_{N} | D_{V50}/ | >3 μm | <1.5 μm |
| Ex. | ratio | (rpm) | D_{V50} (μm) | (μm) | (μm) | (μm) | D_{N} | part. % | vol. % |
| 3a | 80/20 | 12000 | 2.44 | 1.54 | 1.68 | 1.19 | 1.41 | 0.48 | 39.4 |
| 3b | 75/25 | 12000 | 2.53 | 1.66 | 1.73 | 1.18 | 1.47 | 0.62 | 38.3 |
| 3c | 60/40 | 11000 | 3.53 | 1.86 | 2.75 | 1.45 | 1.90 | 4.00 | 13.6 |
| 3d | 60/40 | 12000 | 2.62 | 1.60 | 1.78 | 1.21 | 1.47 | 0.72 | 35.4 |
| 3e | 60/40 | 14000 | 2.36 | 1.60 | 1.59 | 1.13 | 1.41 | 0.36 | 44.7 |
| 3f | 50/50 | 12000 | 2.81 | 1.68 | 2.28 | 1.30 | 1.75 | 2.05 | 22.6 |
| 3g | 40/60 | 11000 | 3.00 | 1.72 | 2.44 | 1.32 | 1.85 | 2.31 | 19.2 |
| 3h | 40/60 | 12000 | 2.88 | 1.75 | 2.07 | 1.27 | 1.63 | 1.45 | 25.8 |
| 3i | 40/60 | 13000 | 2.61 | 1.69 | 1.76 | 1.16 | 1.52 | 0.57 | 37.6 |
| 3j | 40/60 | 14000 | 2.06 | 1.43 | 1.41 | 1.07 | 1.31 | 0.23 | 43.8 |
| 3k | 40/60 | 14500 | 2.39 | 1.67 | 1.64 | 1.15 | 1.43 | 0.49 | 46.5 |
| 3l | 30/70 | 11000 | 3.12 | 1.75 | 2.64 | 1.37 | 1.93 | 2.76 | 16.3 |
| 3m | 30/70 | 12000 | 3.08 | 1.81 | 2.38 | 1.34 | 1.78 | 2.45 | 19.7 |
| 3n | 25/75 | 11000 | 3.15 | 1.85 | 2.46 | 1.31 | 1.88 | 2.15 | 20.7 |
| 3o | 10/90 | 11000 | 3.72 | 2.26 | 3.14 | 1.47 | 2.13 | 4.60 | 12.1 |
| 3p | 5/95 | 11000 | 4.53 | 2.23 | 4.08 | 1.54 | 2.65 | 6.35 | 7.4 |

### Example 4

The same procedure adopted for example 2 is followed, with the only difference that mixtures of DSPA and DPPS with different weight ratios were prepared. The results are summarized in table 4.

TABLE 4

| | | Emulsion | | | Gas-filled microbubbles | | | | |
|---|---|---|---|---|---|---|---|---|---|
| | DSPA/DPPS | Agitation | | D_{N} | D_{V50} | D_{N} | D_{V50}/ | >3 μm | <1.5 μm |
| Ex. | ratio | (rpm) | D_{V50} (μm) | (μm) | (μm) | (μm) | D_{N} | part. % | Vol. % |
| 4a | 25/75 | 12000 | 2.61 | 1.63 | 1.94 | 1.24 | 1.56 | 1.07 | 30.4 |
| 4b | 50/50 | 11000 | 2.81 | 1.86 | 2.35 | 1.39 | 1.69 | 2.67 | 18.4 |
| 4c | 50/50 | 12000 | 2.35 | 1.57 | 1.84 | 1.19 | 1.55 | 0.74 | 34.4 |
| 4d | 75/25 | 12000 | 2.50 | 1.65 | 2.11 | 1.27 | 1.66 | 1.45 | 25.6 |

### Example 5

### Preparations 5a-15i

The same procedure adopted for example 1 is followed, with the only difference that a 1/1 (w/w) phospholipid mixture of DPPG and DSPC has been employed (total concentration 1.0 mg/ml) in admixture with 10% w/w (with respect to the total weight of phospholipid) of palmitic acid. The results are summarized in table 5.

TABLE 5

| | Emulsion | | | Gas-filled microbubbles | | | | |
|---|---|---|---|---|---|---|---|---|
| | Agitation | D_{V50} | D_{N} | D_{V50} | D_{N} | D_{V50}/ | >3 μm | <1.5 μm |
| Ex | (rpm) | (μm) | (μm) | (μm | (μm | D_{N} | part. % | Vol. % |
| 5a | 6000 | 10.02 | 2.64 | 6.87 | 2.07 | 3.32 | 18.00 | 1.8 |
| 5b | 8000 | 5.31 | 2.49 | 3.73 | 1.62 | 2.30 | 7.97 | 7.5 |
| 5c | 9000 | 5.04 | 2.69 | 3.20 | 1.55 | 2.06 | 6.22 | 9.5 |
| 5d | 10000 | 3.82 | 2.02 | 2.85 | 1.38 | 2.07 | 2.65 | 16.4 |
| 5e | 10500 | 3.36 | 1.96 | 2.51 | 1.32 | 1.89 | 2.44 | 20.0 |
| 5f | 11000 | 3.22 | 1.87 | 2.31 | 1.28 | 1.81 | 1.41 | 23.3 |
| 5g | 12000 | 2.69 | 1.61 | 1.74 | 1.14 | 1.53 | 0.52 | 39.2 |
| 5h | 13000 | 2.28 | 1.56 | 1.56 | 1.07 | 1.46 | 0.23 | 47.3 |
| 5i | 14000 | 2.00 | 1.44 | 1.30 | 1.00 | 1.30 | 0.26 | 32.7 |

### Example 6

The same procedure adopted for example 1 is followed, with the only difference that DSEPC is used as phospholipid and perfluorohexane is used as the organic solvent. The applied rotation speed is of 11000 rpm. Dimensions, size distribution and percentage of microbubbles with diameter larger than 3 μm were as follows.

| D_{V50} (μm) | D_{N} (μm) | D_{V50}/D_{N} | >3 μm (%) |
|---|---|---|---|
| 1.65 | 1.11 | 1.49 | 0.30 |

### Example 7

### Preparations 7a-7l

Distilled water (10 ml) containing DPPS (10 mg) as phospholipid is heated at 70° C. for 15 minutes and then cooled at room temperature. 0.8 ml of an organic solvent as specified in the following table 6 were emulsified in this aqueous phase using a high speed homogenizer (Polytron T3000) at 10000 rpm for 1 minute. The emulsion is added to 10 ml of a 15% dextran 15000 solution, frozen and lyophilized (0.2 mbar, 24 hours). After lyophilisation, air is introduced in the lyophilizer. The microbubble suspension obtained after reconstitution with distilled water is analyzed using a Coulter counter. Table 6 summarizes the results in terms of dimensions and size distribution of microbubbles.

TABLE 6

| | | D_{V50} | D_{N} | |
|---|---|---|---|---|
| Ex. | Solvent | (μm) | (μm) | D_{V50}/D_{N} |
| 7a | C₆F₁₄ | 2.77 | 1.44 | 1.92 |
| 7b | CF₃-cyclo-C₆F₁₁ | 2.24 | 1.30 | 1.72 |
| 7c | C₇F₁₆ | 2.48 | 1.40 | 1.77 |
| 7d | C₉F₂₀ | 2.46 | 1.36 | 1.81 |
| 7e | perfluorodecalin | 3.76 | 1.52 | 2.47 |
| 7f | Cyclohexane | 2.61 | 1.41 | 1.85 |
| 7g | Cyclooctane | 2.43 | 1.35 | 1.80 |
| 7h | Decane | 2.01 | 1.12 | 1.79 |
| 7i | Octane | 2.87 | 0.96 | 2.99 |
| 7j | meta xylene | 2.45 | 1.21 | 2.02 |
| 7k | Diisopropyl cetone | 1.83 | 1.05 | 1.74 |
| 7l | CCl₄ | 1.90 | 1.27 | 1.50 |

### Example 8

The above example 7 is repeated with the same methodology, by using perfluoro hexane as the organic solvent and different lyoprotecting agents at different concentrations as outlined in table 7. Table 7 summarizes the results in terms of dimensions and size distribution of microbubbles.

TABLE 7

| | Lyoprotectant and | | | |
|---|---|---|---|---|
| Ex. | concentration (w/w) | D_{V50} (μm) | D_{N} (μm) | D_{V50}/D_{N} |
| 8a | Mannose 5% | 4.35 | 1.90 | 2.29 |
| 8b | Glucose 5% | 2.59 | 0.96 | 2.70 |
| 8c | Sorbitol 5% | 3.84 | 1.40 | 2.74 |
| 8d | Mannitol 10% | 2.22 | 1.22 | 1.82 |
| 8e | Mannitol 5% | 2.24 | 1.21 | 1.85 |
| 8f | Mannitol 4% | 2.54 | 1.45 | 1.75 |
| 8g | Maltose 5% | 3.42 | 0.99 | 3.45 |
| 8h | Dextran 6000 7.5 | 3.30 | 1.48 | 2.23 |
| 8j | Dextran 15000 5% | 2.55 | 1.31 | 1.95 |
| 8k | Dextran 15000 7.5% | 2.77 | 1.44 | 1.92 |
| 8i | Dextran 40000 7.5% | 2.54 | 1.32 | 2.29 |
| 8l | Inulin 5% | 3.58 | 1.43 | 2.70 |

### Example 9

### Preparations 9a-9e

Example 1 is repeated by emulsifying the mixture at a speed of 10000 rpm. In addition, the same example is repeated by adding different amounts of Pluronic F68 (a poloxamer corresponding to Poloxamer 188) into the aqueous phase prior to emulsification, as outlined in table 8. Table 8 shows the results of the comparative experiment, in terms of size distribution and conversion yield of the microbubbles. Conversion yield is given as the percentage number of gas-filled microbubbles formed upon reconstitution of the lyophilized matrix with respect to the number of microdroplets measured in the emulsion.

TABLE 8

| | Pluronic* | | | | |
|---|---|---|---|---|---|
| Example | (mg/ml) | D_{V50} | D_{N} | D_{V50}/D_{N} | Conversion yield (%) |
| 9a | 0 | 2.42 | 1.38 | 1.75 | 28.0 |
| 9b | 0.25 | 4.64 | 1.97 | 2.36 | 18.8 |
| 9c | 0.5 | 13.85 | 1.38 | 10.04 | 7.3 |
| 9d | 1.0 | 12.59 | 1.49 | 8.45 | 3.2 |
| 9e | 2.0 | 15.80 | 1.23 | 12.85 | 0.5 |

*Concentration referred to the volume of aqueous phase

The above results show that with a concentration of poloxamer corresponding to half the concentration of the phospholipid (i.e. about 33% of the total amount of surfactants in the mixture), both conversion yields and size distribution of microbubbles are negatively affected.

### Example 10

### Preparations 10a-10d

Example 9 is repeated, but instead of adding Pluronic F68 to the aqueous phase, different amounts of cholesterol (from Fluka) were added to the organic phase, prior to emulsification, as outlined in table 9. Table 9 shows the results of the comparative experiment, in terms of size distribution and conversion yield (from the microdroplets of the emulsion) of the microbubbles.

TABLE 9

| | | | | | |
|---|---|---|---|---|---|
| | Cholesterol* | | | | Conversion |
| Example | (mg/ml) | D_{V50} | D_{N} | D_{V50}/D_{N} | yield (%) |

| | | | | | |
|---|---|---|---|---|---|
| 10a | 0 | 2.42 | 1.38 | 1.75 | 28.0 |
| 10b | 0.10 | 3.79 | 1.31 | 2.89 | 17.8 |
| 10c | 0.25 | 1.35 | 1.05 | 1.28 | 5.7 |
| 10d | 0.50 | 14.02 | 1.70 | 8.25 | 0.8 |

*Concentration referred to the volume of the aqueous phase

The above results show that with a concentration of 0.050% (w/w) of cholesterol in the aqueous phase, both conversion yield and size distribution of microbubbles are highly negatively affected. A concentration of 0.025%, while it may provide acceptable dimensions and size distribution of microbubbles, still results in a rather low conversion yield.

### Example 11

Distilled water (30 ml) containing 60 mg of DPPS and 3 g of mannitol is heated to 70° C. during 15 minutes then cooled to room temperature. Perfluoroheptane is emulsified in this aqueous phase using a high speed homogenizer (Polytron®, 12500 rpm, 1 minute).

The resulting emulsion, showing a median diameter in volume (D_{V50}) of 2.3 μm and a mean diameter in number (D_{N}) of 2.0 μm, is washed once by centrifugation, resuspended in 30 ml of a 10% solution of mannitol in distilled water and then divided in three portions (3×10 ml).

The first portion (A) is used as such for the subsequent lyophilization step. The second portion (B) is collected into a syringe and hand-injected through a 5 μm Nuclepore® filter (47 mm—Polycarbonate). The third portion (C) is filtered through a 3 μm Nuclepore® filter (47 mm—Polycarbonate) with the same method.

The emulsions were frozen in 100 ml balloon (−45° C. for 5 minutes) then freeze dried (0.2 mBar, for 72 hours).

Atmospheric pressure is restored by introducing a 35/65 mixture of C₄F₁₀ and air. The respective lyophilisates were dispersed in distilled water (10 ml). The so obtained microbubbles suspensions are analysed using a Coulter counter and the results are reported in the following table

| | D_{V50} | D_{N} | D_{V50}/D_{N} | <1.5 μm |
|---|---|---|---|---|
| Part A | 1.71 | 1.12 | 1.53 | 40.2 |
| Part B | 1.65 | 1.12 | 1.47 | 42.3 |
| Part C | 1.57 | 1.09 | 1.44 | 46.3 |

As shown by the above results, the additional filtration step allows to further reduce the dimension of the microbubbles and to reduce the respective size distribution.

### Example 12

Example 1 has been repeated, by using 10 mg of a 7/3 (w/w) mixture of DSPC/DSTAP, at an agitation speed of 11000 rpm.

Characterization of emulsion droplets and microbubbles were as follows:

| | |
|---|---|
| Emulsion | |
| droplets | Gas-filled microbubbles |

| D_{V50} | D_{N} | D_{V50} | D_{N} | >3 μm | <1.5 μm |
|---|---|---|---|---|---|
| 2.36 | 1.48 | 2.10 | 1.12 | 0.63 | 32.7 |

### Example 13

The preparation of example 1 is repeated, by emulsifying the mixture at a speed of 10000 rpm (example 13a).

The same preparation is repeated, by further adding about 0.9 mg of DSPE-PEG2000 (about 8.3% of the total amount of dispersed phospholipids) to the initial aqueous suspension (example 13b).

No washing by centrifugation is performed on either the two preparations. Table 10 shows the characterization of the two preparations, both of the emulsion and of the microbubbles suspension.

TABLE 10

| | Emulsion | | Microbubbles | | | |
|---|---|---|---|---|---|---|
| | | D_{N} | D_{V50} | D_{N} | | Conversion |
| Example | D_{V50} (μm) | (μm) | (μm) | (μm) | D_{V50}/D_{N} | Yield (%) |
| 13a | 3.19 | 1.66 | 2.66 | 1.33 | 2.00 | 29.5 |
| 13b | 4.32 | 1.43 | 5.81 | 1.18 | 4.92 | 18.8 |

The above results show that with a concentration of DSPE-PEG of less than 10% by weight (with respect to the total amount of phospholipids), both conversion yields and size distribution of microbubbles are negatively affected.

### Example 14

The preparation of example 11 is repeated, by replacing DPPS with the same amount of a 1:1 (w/w) mixture of DAPC/DPPS.

The resulting emulsion is divided in three portions of 10 ml, without washing it by centrifugation.

Aqueous suspensions of DSPE-PEG2000 and of DSPE-PEG5000 are separately prepared by dispersing 25 mg of the respective DSPE-PEG in 5 ml of a 10% mannitol solution under sonication (3 mm sonication probe, Branson 250 sonifier, output 30%, for 5 min).

An aliquot of 2.5 ml of a 10% mannitol solution is then added to a first portion of the emulsion (example 14a)

An aliquot of 2.5 ml of the prepared DSPE-PEG2000 suspension is added to a second portion of the emulsion (example 14b)

An aliquot of 2.5 ml of the prepared DSPE-PEG5000 suspension is added to a third portion of the emulsion (example 14c)

The three mixtures are heated at 60° C. under stirring for one hour. After cooling at room temperature, the size of microdroplets are determined by means of Malvern Mastersizer. Results are reported in table 11.

The emulsions are then freeze dried according to the procedure of example 11. Atmospheric pressure is restored by introducing a 35/65 mixture of C₄F₁₀ and air. The respective lyophilisates were dispersed in distilled water (10 ml). The so obtained microbubbles suspensions were analysed using a Coulter counter (see table 11).

Microbubble suspensions are then washed twice with distilled water by centrifugation (180 g/10 min) and lyophilized again according to the above procedure. The amount of DSPE-PEG in the dried composition is determined by means of HPLC-MS. Results are given in the following table 11.

TABLE 11

| | Emulsion | | | | |
|---|---|---|---|---|---|
| | D_{V50} | D_{N} | Microbubbles | | |
| Example | (μm) | (μm) | D_{V50} (μm) | D_{N} (μm) | DSPE-PEG (% w/w) |
| 14a | 2.6 | 2.3 | 1.9 | 1.1 | 0.0 |
| 14b | 2.5 | 2.3 | 3.4 | 1.3 | 35.5 |
| 14c | 2.5 | 2.3 | 2.2 | 1.2 | 37.9 |

As inferable from the above results, the subsequent addition of a DSPE-PEG suspension to the formed emulsion allows introducing relatively high amounts of DSPE-PEG in the composition of the stabilizing layer (in this case more than 30% of the total weight of the phospholipids forming the stabilizing envelope), without negatively affecting the final properties of the microbubbles.

Similar results can be obtained with other PEG-modified phospholipids, in particular DSPE-PEG2000-Biotin or DSPE-PEG2000-mal, and with peptide bearing phospholipids, in particular DSPE-PEG2000-mal-SATA-RGD4C. This latter peptide bearing phospholipid can be prepared according to known techniques, by reacting the RGD-4C peptide with SATA, deprotecting the thiol group of SATA and reacting the deprotected RGD4C-SATA with DSPE-PEG2000-mal. The preparation method described in “Development of EGF-conjugated liposomes for targeted delivery of boronated DNA-binding agents”, by Bohl Kullberg et al., Bioconjugate chemistry 2002, 13, 737-743, (describing the insertion of a EGF protein in a DSPE-PEG-mal molecule), can be conveniently used.

### Example 15

20 mg of a 80/20 (w/w) DSPC/DSPA mixture are dissolved in 1.6 ml of cyclooctane at 80° C. and the suspension is added to 20 ml of distilled water containing 10% (w/w) of PEG4000 (Fluka). The mixture is emulsified by using a high speed homogenizer (PolytronT3000) for 1 minute at 8000 rpm.

DSPE-PEG1000 (0.29 μmole) and DSPE-PEG2000-mal-SATA-RGD4C (0.29 μmole) are dissolved in EtOH/water 9/1 v/v; after evaporation, the obtained lipid film is dried overnight at 25° C. and 0.2 mBar and resuspended in 320 μl of water at 60° C.

The obtained solution is added to the emulsion previously prepared and the resulting emulsion is heated under stirring at 80° C. for 1 hour, followed by cooling at to room temperature. The emulsion is then centrifuged (1300 g/10 min) to eliminate the excess of phospholipid and the floating microdroplets are recovered and resuspended in 40 ml of PEG4000 10% solution.

The resulting emulsion was sampled in DIN8R vials (1 ml per vial) and the vials are frozen at −50° C. for 2 hours (Christ Epsilon lyophilizer), then freeze-dried at −25° C. and 0.2 mBar for 12 hours, with a final drying step at 30° C. and 0.05 mBar for 6 hours.

The lyophilized product is then exposed to an atmosphere containing 35% of perfluoro-n-butane and 65% of nitrogen and the vials are sealed.

The product is finally dispersed in a volume of water twice than the initial one by gentle hand shaking.

Similar results are obtained when the DSPC/DSPA mixture is replaced by a 80/20 DSPC/Stearic acid mixture.

### Example 16

10 mg of a 1:1 (w/w) DPPS/DSPC mixture are added to about 10 ml of a 10% (w/w) mannitol aqueous solution.

The mixture is heated at 70° C. for 15 minutes and then cooled at room temperature (22° C.). Cyclooctane is added at a flow rate 0.2 mL/min through an inlet of a micromixer (standard slit Interdigidital micro Mixer, housing SS 316Ti with nickel-on-copper inlay, 40 μm×300 μm, Institut für Microtechnik Mainz GmbH) to the aqueous phase circulating at 20 ml/min at room temperature, for a total amount of 7.4% (v/v) of organic solvent. Upon completion of the addition of the organic solvent, the emulsion is recirculated in the micromixer for additional 20 minutes.

The emulsion is then divided into five aliquots of 2 ml each and it is introduced into five vials DIN8R. Four vials are sealed and heated for 30 minutes at temperatures of 60, 80, 100 and 120° C., respectively, as indicated in table 12, while the fifth is not heated.

The emulsions are then cooled to room temperature and the content of the five vials is subjected to lyophilization according to the following procedure. 1 ml of each emulsion is collected into a DIN8R vial and frozen at −5° C.; the temperature is lowered to −45° C. during 1 hour and the emulsion is then freeze-dried at −25° C. and 0.2 mbar during 12 hours (Telstar Lyobeta35 lyophilizer), with a final drying step at 30° C. and 0.2 mbar for 5 hours.

The lyophilized product is then exposed to an atmosphere containing 35% of perfluoro-n-butane and 65% of nitrogen and then dispersed in a volume of water twice than the initial one by gentle hand shaking. Table 12 shows the result of the characterization of the final suspension of microbubbles.

TABLE 12

| | | | | Number of μbubbles |
|---|---|---|---|---|
| Heating | D_{V50} | D_{N} | D_{V50}/D_{N} | per ml of emulsion |
| no heating | 10.45 | 1.63 | 6.41 | 5.34 × 10⁷ |
| 60° C. | 4.85 | 1.32 | 3.67 | 7.83 × 10⁷ |
| 80° C. | 5.34 | 1.29 | 4.14 | 8.51 × 10⁷ |
| 100° C. | 6.96 | 1.66 | 4.19 | 4.92 × 10⁸ |
| 120° C. | 3.05 | 1.50 | 2.03 | 8.69 × 10⁸ |

From the above results, it appears that by subjecting the formed emulsion to a thermal treatment allows to narrow the size distribution of the final microbubble suspension, while also increasing the total number of microbubbles. In particular, by increasing the heating temperature above 100° C., it is possible to obtain a relatively narrow size distribution of microbubbles also in the absence of any washing step of the emulsion, as well as an increase of the total number of microbubbles in the suspension.

### Example 17

The 20 mg of 80/20 (w/w) DSPC/DSPA phospholipid mixture are dissolved in 1.6 ml of cyclooctane at 80° C. and the suspension is added to 20 ml of distilled water containing 10% (w/w) of PEG4000 (Fluka).

The mixture is emulsified by using a high speed homogenizer (PolytronT3000) for 1 minute at 8000 rpm.

The resulting emulsion is heated under stirring at 80° C. for 1 hour then cooled to room temperature. Afterwards it is centrifuged (1300 rpm for 10 min) to eliminate the excess of phospholipid and the floating microdroplets are recovered and resuspended in 40 ml of PEG4000 10% solution.

The resulting emulsion was sampled in DIN8R vials (1 ml per vial) and the vials are frozen at −50° C. for 2 hours (Christ Epsilon lyophilizer), then freeze-dried at −25° C. and 0.2 mBar for 12 hours, with a final drying step at 30° C. and 0.05 mBar for 6 hours.

The lyophilized product is then exposed to an atmosphere containing 35% of perfluoro-n-butane and 65% of nitrogen and the vials are sealed.

The product is finally dispersed in a volume of water twice than the initial one by gentle hand shaking.

Table 13 shows the characterization of of the emulsion and of the microbubbles suspension.

TABLE 13

| Emulsion | | Microbubbles | | | |
|---|---|---|---|---|---|
| D_{V50} (μm) | D_{N} (μm) | D_{V50} (μm) | D_{N} (μm) | D_{V50}/D_{N} | <1.5 μm |
| 2.89 | 1.66 | 2.51 | 1.27 | 1.98 | 22.1 |

### Example 18

Distilled water (10 ml) containing 10 mg of DPPS and 1 g of mannitol is heated to 70° C. during 15 minutes then cooled to room temperature. DPPE-MPB (1,2-Dipalmitoyl-sn-glycero-3-phosphoethanolamine-N-[4-(p-maleimidophenyl) butyramide] Na salt—Avanti Polar Lipids) is added (4.8% by weight—0.5 mg). This phospholipid is dispersed in the aqueous phase using a ultrasound bath (Branson 1210—3 minutes).

Perfluoroheptane (0.8 ml from Fluka) is emulsified in this aqueous phase (cooled with a ice bath) using a high speed homogenizer (Polytron® T3000, 15000 rpm, 1 minute).

The resulting emulsion showed a median diameter in volume (D_{V50}) of 2.3 μm and a mean diameter in number (D_{N}) of 2.1 μm as determined with a Malvern Mastersizer.

The emulsion is washed twice by centrifugation then resuspended in 9.5 ml of a 10% solution of mannitol in distilled water. The washed emulsion is frozen (−45° C., 5 minutes) then freeze dried (under 0.2 mBar, for 24 hours).

Atmospheric pressure is restored by introducing a 35/65 mixture of C₄F₁₀ and air. The lyophilisate is dispersed in distilled water (20 ml), microbubbles were washed once by centrifugation and then redispersed in 4 ml of an EDTA containing phosphate buffered saline (molar composition: 10 mM phosphate, 2.7 mM KCl, 137 mM NaCl, 10 mM EDTA), containing 3.4 mg of thioacetylated avidin, 400 μl of a hydroxylamine solution (13.92 mg in PBS 50 mM, pH: 7.5) were added to deprotect the thiol group of the thioacetylated avidin.

The suspension is stirred by inversion on a disk rotator (Fisher Scientific) for 2 hours. Then 150 μl of NaOH 1N were added.

The so obtained avidin-labelled microbubbles were washed twice with PBS by centrifugation (10000 rpm, 10 minutes, Sigma centrifuge 3K10). The microbubbles suspension obtained is analysed using a Coulter counter showing a D_{V50} diameter of 1.6 μm and a D_{N} of 1.2 μm.

The efficacy of targeted microbubbles composition was tested both in vitro and in vivo.

In vitro Experiment:

To test the effective bonding of acetylated avidin to the surface of the microbubbles, two sets of fibrin containing wells were prepared. In the first set, only a fibrin surface is present. In the second set, the fibrin is pre-treated with a biotin-labelled antifibrin peptide (DX-278, disclosed in WO 02/055544). Microbubble suspensions prepared as above were added to the wells (5×10⁸ microbubbles/well). After 2 hours of incubation (upside down) and several washings, the fibrin surfaces in the two set of wells were observed by means of an optical microscope. While essentially no microbubble could be observed in the wells without the biotinylated antifibrin peptide, a massive coverage of microbubbles weas observed in the biotinylated antifibrin peptide containing wells.

In vivo Experiment:

A thrombus is formed in the abdominal aorta of two rabbits by the FeCl₃ method (Lockyer et al, 1999, Journal of Cardiovascular Pharmacology, vol 33, pp 718-725).

Echo imaging is performed with an HDI 5000 ultrasound machine (Philips), pulse inversion mode, L7-4 probe, MI: 0.07.

A biotinylated antibody (CD41 specific for the GPIIB/IIIA receptor of activated platelets) is then injected intravenously to the two rabbit.

After 30 minutes, the microbubble suspension comprising avidin-labelled microbubbles is injected intravenously (1×10⁹ microbubbles/ml) in the first rabbit. Fifteen minutes after the injection, a strong opacification of the thrombus is observed for the suspension. This opacification is still visible after at least one hour from the injection.

The same amount of the microbubble suspension without avidin-labelled microbubbles is injected intravenously in the second rabbit. Only a light opacification of the thrombus is observed.

### Example 19

### Preparation of Microbubbles Containing Abcximab Targeting Antibody

20 mg of a mixture of DSPC/Stearate (80/20 by moles) were dissolved in cyclooctane (1.6 mL) at 80° C. Separately, 19 mL of PEG4000 10% solution were added to 1 mL of phosphate buffer 62.5 mM pH 7.5. The organic phase containing the phospholipids was then added to the aqueous phase and emulsified by using a high speed homogenizer (Polytron T3000) for 1 min (9000 rpm), to obtain an emulsion.

In a separate vessel, 1.9 mg of DSPE-PEG2000-mal were dissolved in ethanol (0.4 mL); after solvents evaporation, the lipid film obtained was dried overnight at 25° C. and 0.2 mBar and dispersed in 370 μL of phosphate buffer 62.5 mM pH 7.5 at 60° C., to obtain a micellar suspension of DSPE-PEG2000-mal.

The micellar suspension was added to the emulsion and the resulting emulsion was heated under stirring at 60° C. for 1 h, then cooled at room temperature (about 22° C.).

An aqueous suspension (2 mg/mL) of ReoPro® Fab antibody (abciximab, from Eli Lilly) was reduced with 1 mM TCEP, 50 mM Tris HCl/50 mM EDTA pH 6.8, for 1 h at 37° C. according to the procedure described in patent WO 2004/043492. Traces of radiolabeled ReoPro (with ¹²⁵I) were also added to the mixture, for the subsequent determination of the density of the antibody bound to the surface of the microbubbles. The solution was spun through a 5 mL spin-column at 1000 g (Zeba spin column, Pierce) equilibrated in phosphate buffer 5 mM, pH 7.4. Different volumes (see table 14) of the obtained phosphate buffer solution containing reduced Fab antibody (at a concentration of about 1.5 mg/mL) were immediately added to 10 mL of the emulsion and the resulting suspension was mixed at 22° C. for 2 h. The emulsion was finally diluted twice in 20 mL of 10% PEG4000 solution and sampled in DIN8R vials (1 mL per vial).

Vials were frozen at −50° C. for 2 h (Christ Epsilon lyophilizer), then freeze-dried at −25° C. and 0.2 mBar. The lyophilized product was then exposed to an atmosphere containing 35% of perfluoro-n-butane and 65% of air. The vials were sealed. The product was dispersed in a volume of water twice the initial one (i.e. 2 mL) by gentle hand shaking.

The suspension of microbubbles was washed twice (centrifuge at 180 g, 10 min) and the supernatant containing the microbubbles was resuspended in 1 mL of saline. Number and volume of microbubbles were determined by Coulter counter measurement, and the total surface of the microbubbles was calculated.

The density of the antibody bound to the surface of the microbubbles was then determined by measuring the radioactive response of the suspension (by means of Auto-Gamma Cobra II-Packard), converting said value into the corresponding number of molecules of antibody and dividing said number by the total surface of the microbubbles, as determined by the Coulter counter measurement.

Table 14 shows the results, indicating the volume of the solution containing the reduced Fab antibody (at a concentration of 1.5 mg/mL, 32 nmoles/mL) and the respective density of antibody on the surface of the microbubbles.

TABLE 14
Density of antibody on the surface of the microbubbles

| Preparation | Volume of antibody | Density of antibody |
|---|---|---|
| no. | suspension (μl) | (molecules/μm²) |
| 19a | 435 | 13000 |
| 19b | 185 | 4500 |
| 19c | 60 | 1700 |
| 19d | 25 | 500 |
| 19e | 14 | 200 |
| 19f | 5 | 100 |
| 19g | 1.5 | 25 |

### Example 20

### In vitro Binding Activity of Abcximab-Targeted Microbubbles

To test the effective binding of these conjugates, targeted microbubbles prepared according to example 19 were injected in a flow chamber set up comprising human platelets. Microbubbles (at equivalent surface of 2×10⁹ μm²/14 mL) were drawn through the flow chamber (FCS2, Bioptech, USA) and their adhesion onto the human platelet layer was assessed for 10 min at a flow rate of 2 mL/min (160 s⁻¹) in the presence of 50% human plasma in PBS (v:v, Biomeda collected on citrate, ref. ES1020P, Stehelin & Cie AG). Expression of GPIIb/IIIa by activated platelets was shown by immunohistochemistry assay. A quantitative analysis of microbubble accumulation was performed by counting the number of microbubbles adhering in the observed area at 2 min intervals over the total 10 min infusion, using the image processing program Analysis FIVE (SIS, Germany). After 10 min, five pictures were taken randomly and averaged then divided by ten, representing the rate of microbubble accumulation per minute (RMA/min). Each observed area was 183×137 μm, as measured with the aid of a stage micrometer. Imaging was performed between the middle and the exit of the chamber. Results are provided in the following table 15.

TABLE 15
Rate of Microbubbles Accumulation per minute (RMA/min)

| | Preparation no. | | | | | | |
|---|---|---|---|---|---|---|---|
| | 19a | 19b | 19c | 19d | 19e | 19f | 19g |
| (RMA/min) | 16 | 15.7 | 14 | 13.4 | 5.1 | 1.7 | 0.2 |

### Example 21

### Preparation of Streptavidin-Microbubbles

60 mg of a mixture of DSPC/DSPG/Palmitic acid (41/34/25 by moles) were dissolved in cyclooctane (4.8 mL) at 70° C. Separately, 57 mL of PEG4000 10% solution were added to 3 mL of phosphate buffer 100 mM pH 6.0. The organic phase containing the phospholipids was then added to the aqueous phase and emulsified by using a high speed homogenizer (Megatron MT3000) for 4 min (12000 rpm), to obtain an emulsion.

In a separate vessel, 1.9 mg of DSPE-PEG2000-mal were dissolved in ethanol (0.4 mL); after solvents evaporation, the lipid film obtained was dried overnight at 25° C. and 0.2 mBar and dispersed in 200 μL of phosphate buffer 100 mM pH 6.0 at 60° C., to obtain a micellar suspension of DSPE-PEG2000-mal.

The micellar suspension was added to the emulsion and the resulting emulsion was heated under stirring at 60° C. for 1 h, then cooled at room temperature (about 22° C.).

A water suspension (10 mg/mL) of Streptavidin (IBA Germany) was reacted with 1 mM Sulfo-LC-SDPD ((Sulfosuccinimidyl 6-[3′-(2-pyridyldithio)propionamido]-hexanoate), Pierce) in 50 mM phosphate buffer 150 mM NaCl pH 7.4, for 40 min at room temperature. The solution was spun through a 5 mL spin-column at 1000 g (Zeba spin column, Pierce, #89891) equilibrated in phosphate buffer 5 mM pH7.4. The functionalized Streptavidin was then reduced with 1 mM TCEP, 50 mM Tris HCl/5 mM EDTA pH 6.8, for 15 min at room temperature. The reduced Streptavidin was spun through a 5 mL spin-column at 1000 g. Different volumes (see table 16) of the solution containing the reduced streptavidin (at a concentration of about 1.6 mg/ml (or 30 nmoles/mL) was added to 10 mL of the emulsion and the resulting mixture was agitated at 22° C. for 2 h. The obtained emulsion was finally diluted twice in 20 mL of 10% PEG4000 solution sampled in DIN4R vials (300 μL per vial).

Vials were frozen at −50° C. for 2 h (Christ Epsilon lyophilizer), then freeze-dried at −25° C. and 0.2 mBar. The lyophilized product was then exposed to an atmosphere containing 35% of perfluoro-n-butane and 65% of air. The vials were sealed. The product was dispersed in a volume of saline (700 μL, 150 mM NaCl) by gentle hand shaking.

The content of one vial was reconstituted in 1 mL of saline (0.9% NaCl), and incubated with 1.6 nmole of ¹⁴C-biotin for 10 min at room temperature. The unbound biotin was removed by repeated centrifugations (180 g, 10 min) of the bubbles. After the second centifugation, the supernatant—comprising the bubbles—was collected, characterized by Coulter counter measurement (bubble concentration, total bubble surface/mL and total bubble volume/mL) and the radioactivity was counted (Liquid scintillation analyzer—Beta Tris Carb 2200CA—Packard). The extent of ¹⁴C-biotin binding to a streptavidin standard, after gel filtration (Zeba Desalt spin column, Pierce), was used as a reference for the calculation of the streptavidin density (expressed as molecules/μm²) on the surface of the microbubbles. The antibody binding capacity was determined by incubating a ¹²⁵I-labelled biotinylated IgG antibody with the streptavidin microbubbles for 10 min at room temperature. The microbubbles suspension was then treated as described in Example 19, to characterize the microbubbles by means of coulter counter and determining the density of streptavidin molecules on the surface of microbubbles. Results are provided in the following table 16.

TABLE 16
Density of streptavidin molecules on the surface of the microbubbles

| | Volume of streptavidin | Density of streptavidin |
|---|---|---|
| Preparation no. | suspension (μL) | (molecules/μm²) |
| 21a | 530 | 16500 |
| 21b | 260 | 7300 |
| 21c | 200 | 3800 |
| 21d | 100 | 1700 |

### Example 22

### Attachment of Biotin Anti-Flk-1 Antibody onto Streptavidin-Microbubbles

300 μL of a solution of biotin anti-Flk-1 antibody (supplied by eBiosciences) with different concentrations of antibody (see table 17) were added into vials containing a suspension of streptavidin-microbubbles prepared according to example 21. After 10 minutes under agitation, the preparation was ready for the binding test. The following table 17 shows the amount of antibody added to the respective preparation of example 21, to obtain the preparations according to this example.

TABLE 17
Preparation of anti-Flk-1 targeted microbubbles
with biotin-streptavidin binding

| | Streptavidin preparation of | concentration of anti- |
|---|---|---|
| Preparation no. | example 21 | Flk-1 antibody (μg/ml) |
| 22a | 21a | 40 |
| 22b | 21b | 15 |
| 22c | 21c | 10 |
| 22d | 21d | 5 |

### Example 23

### Determination of the Density of Anti-Flk-1 Antibody on the Surface of Microbubbles

The preparation of example 22 was repeated to determine the density of the antibody bound to the surface of the microbubbles. To this end, traces of ¹²⁵I-labeled biotin anti-Flk-1 were added to the solution of the unlabeled antibody to be added at different concentrations into the vials. Then, after treatment according to example 19, the microbubbles were characterised by Coulter counter measurement and the density of streptavidin molecules on the surface of microbubbles was determined as described in said example. The results are given in the following table 18.

TABLE 18
Density of anti-Flk-1 antibody on the surface of the microbubbles

| Preparation | Initial streptavidin | Concentration of | Density of antibody |
|---|---|---|---|
| no. | preparation | antibody (μg/mL) | per μm² |
| 22a | 21a | 40 | 9700 |
| 22b | 21b | 15 | 6000 |
| 22c | 21c | 15 | 2700 |
| 22d | 21d | 10 | 1100 |

### Example 24

### In vitro Binding Activity of Anti-Flk-1 Targeted Microbubbles

Suspensions of targeted microbubbles prepared according to example 22 were injected in a flow chamber as described in example 20, and their binding activity determined according to the procedure described in said example.

Results are provided in the following table 19.

TABLE 19
Rate of Microbubbles Accumulation per minute (RMA/min)

| | Preparation no. | | | | |
|---|---|---|---|---|---|
| | 22a | 22b | 22c | 22d | 22e |
| (RMA/min) | 5.1 | 5.0 | 4.7 | 4.3 | 4.0 |

### Example 25

### Attachment of Biotin Anti-P-Selectin Antibody onto Streptavidin-Microbubbles

300 μL of a 15 μg/ml solution of biotin anti-P-Selectin antibody (supplied by BD-Pharmingen) was added into vial containing a suspension of streptavidin-microbubbles prepared according to example 21. After 10 minutes under agitation, the preparation was ready for the binding test. The following table 20 shows the amount of antibody added to the respective preparation of example 21, to obtain the preparations according to this example.

### Example 26

### Determination of the Density of Anti-P-Selectin Antibody on the Surface of Microbubbles

The preparation of example 25 was repeated to determine the density of the antibody bound to the surface of the microbubbles. To this end, traces of ¹²⁵I-labeled biotin anti-P-Selectin were added to the solution of the unlabeled antibody to be added at different concentrations into the vials. Then, after two washing steps, the microbubbles were characterised by Coulter counter measurement and the associated radioactivity was measured as described in example 19, to obtain a density of about 6000 antibodies molecules per μm² of microbubbles surface.

### Example 27

### In vitro Binding Activity of Anti-P-Selectin Targeted Microbubbles

Suspensions of targeted microbubbles prepared according to example 25 were injected in a flow chamber as described in example 20, and their binding activity determined according to the procedure described in said example. A value of 5.1 of RMA/min (rate of microbubbles accumulation per minute) was determined.

1. A method for preparing an aqueous suspension of gas-filled microbubbles comprising a ligand agent, which comprises the steps of
a) preparing an aqueous-organic emulsion comprising i) an aqueous medium, ii) an organic solvent substantially immiscible with water, iii) an amphiphilic material comprising a phospholipid and iv) a lyoprotecting agent;
b) adding a precursor of said ligand agent with said aqueous-organic emulsion;
c) converting said precursor into said ligand agent;
d) lyophilizing said mixture, to obtain a lyophilized matrix;
e) contacting said lyophilized matrix with a biocompatible gas; and
f) reconstituting said lyophilized matrix by dissolving it in a physiologically acceptable aqueous carrier liquid, to obtain said suspension of gas-filled microbubbles.

2. A method according to claim 1, wherein said precursor comprises a phospholipid.

3. A method according to claim 1, wherein said precursor comprises a hydrophilic polymer covalently bound to said phospholipid.

4. A method according to any one of claims 2 or 3, wherein said phospholipid is a phosphoethanolamine.

5. A method according to any one of claims 2 or 3, wherein said hydrophylic polymer is a polyethyleneglycol.

6. A method according to claim 1, wherein said precursor is added in a molar amount of from about 0.1% to about 10% of a molar amount of said amphiphilic material.

7. A method according to claim 1, wherein step c comprises adding a compound comprising the ligand agent to the emulsion comprising said precursor.

8. A method according to claim 7, wherein the compound comprising the ligand agent is added in a molar ratio of from about 1:2 to about 1:10000 with respect to the precursor in the emulsion.

9. A method according to claim 8, wherein said molar ratio is of from about 1:5 to 1:1000.

10. A method according to claim 1, wherein said ligand agent comprises a targeting ligand.

11. A method according to claim 10, wherein said targeting ligand is an antibody or a fragment thereof.

12. A method according to claim 1 wherein said ligand agent comprises a moiety of an affinity binding pair.

13. A method according to claim 12 wherein said moiety is avidin or streptavidin.

14. A method according to any one of claims 12 or 13, which comprises the further step of adding a targeting ligand, comprising a complementary moiety of said affinity binding pair, to the suspension of gas-filled microbubbles obtained in step f).

15. A method according to claim 14, wherein the targeting ligand is an antibody or a fragment thereof.

16. A method according to claim 14, wherein said targeting ligand is added in a molar ratio of from about 1:2 to about 20:1 with respect to the ligand agent.

17. A method according to claim 1 wherein said biocompatible gas comprises SF₆ or a perfluorocarbon, optionally in admixture with air, nitrogen, oxygen or carbon dioxide.

18. A method according to claim 1 wherein said perfluorocarbon is perfluoropropane or perfluorobutane.

19. A method for preparing a lyophilized precursor of gas-filled microbubbles comprising a ligand agent, comprising the steps of
a) preparing an aqueous-organic emulsion comprising i) an aqueous medium, ii) an organic solvent substantially immiscible with water, iii) an amphiphilic material comprising a phospholipid and iv) a lyoprotecting agent;
b) adding a precursor of the ligand agent to said aqueous-organic emulsion;
c) converting said precursor of the ligand agent into said ligand agent;
d) lyophilizing said mixture, to obtain a lyophilized matrix containing said precursor of gas-filled microbubbles.

20. A method according to claim 19, wherein said precursor of the ligand agent comprises a phospholipid.

21. A method according to claim 19, wherein said precursor of the ligand agent comprises a hydrophilic polymer covalently bound to said phospholipid.

22. A method according to any one of claims 20 or 21, wherein said phospholipid is a phosphoethanolamine.

23. A method according to any one of claims 20 or 21, wherein said hydrophylic polymer is a polyethyleneglycol.

24. A method according to claim 19, wherein said precursor of the ligand agent is added in a molar amount of from about 0.1% to about 10% of a molar amount of said amphiphilic material.

25. A method according to claim 19, wherein step c comprises adding a compound comprising the ligand agent to the emulsion comprising said precursor of the ligand agent.

26. A method according to claim 21, wherein the compound comprising the ligand agent is added in a molar ratio of from about 1:2 to about 1:10000 with respect to the precursor in the emulsion.

27. A method according to claim 19, wherein said ligand agent comprises a targeting ligand.

28. A method according to claim 27, wherein said targeting ligand is an antibody or a fragment thereof.

29. A method according to claim 19, wherein said ligand agent comprises a moiety of an affinity binding pair.

30. A method according to claim 29 wherein said moiety is avidin or streptavidin.

31. A method according to any one of claims 1 or 19, wherein said precursor of the ligand agent is converted into the ligand agent before adding said precursor to the aqueous-organic emulsion.

32. A method according to any one of claims 1 or 19, wherein said precursor of the ligand agent is added to said aqueous-organic emulsion in the form of an aqueous micellar suspension of said ligand agent.

33. A method according to claim 32 wherein said precursor of the ligand agent is converted into the ligand agent in said micellar suspension, before the addition thereof to the aqueous-organic emulsion.

34. A method for preparing a suspension of gas-filled microbubbles comprising a targeting ligand bound to an amphiphilic compound by means of an affinity binding pair, which comprises the steps of:
a) contacting a lyophilized precursor of gas-filled microbubbles, obtained according to method comprising the steps of:
preparing an aqueous-organic emulsion comprising i) an aqueous medium, ii) an organic solvent substantially immiscible with water, iii) an amphiphilic material comprising a phospholipid and iv) a lyoprotecting agent;
adding a precursor of a moiety of an affinity binding pair to said aqueous-organic emulsion;
converting said precursor of the moiety of the affinity binding pair into said moiety; and
lyophilizing said mixture, to obtain said lyophilized precursor;
with a biocompatible gas;
b) reconstituting said lyophilized precursor by dissolving it in a physiologically acceptable aqueous carrier liquid, to obtain said suspension of gas-filled microbubbles;
c) adding a targeting ligand, comprising a complementary moiety of said affinity binding pair, to the suspension of gas-filled microbubbles.

35. A method according to claim 15, wherein said targeting ligand is added in a molar ratio of from about 1:2 to about 20:1 with respect to the ligand agent.
